(19) United States
(12) Patent Application Publication
(10) Pub. No.: US 2025/0250233 A1
(43) Pub. Date: Aug. 7, 2025

(54) PROCESSES AND INTERMEDIATES FOR THE PREPARATION OF PRIDOPIDINE

(71) Applicant: PRILENIA NEUROTHERAPEUTICS LTD., Yakum (IL)

(72) Inventors: Eliezer BAHAR, Tel Aviv (IL); Elena BERELOVICH, Hadera (IL)

(73) Assignee: PRILENIA NEUROTHERAPEUTICS LTD., Yakum (IL)

(21) Appl. No.: 18/856,905

(22) PCT Filed: May 2, 2023
(86) PCT No.: PCT/IL2023/050452

§ 371 Date: Oct. 15, 2024

Related U.S. Application Data
(60) Provisional application No. 63/337,684, filed on May 3, 2022.

(51) Int. Cl. C07D 211/24; B01J 23/30; B01J 23/42; B01J 23/44; B01J 23/46; B01J 31/22; C07D 213/32

(52) U.S. Cl. CPC C07D 211/24; B01J 23/30; B01J 23/42; B01J 23/44; B01J 23/462; B01J 31/2295; C07D 213/32

(57) ABSTRACT

Provided herein processes and intermediates for the preparation of pridopidine.

### FIELD OF THE INVENTION

This invention is directed to processes and intermediates for the preparation of pridopidine.

### BACKGROUND OF THE INVENTION

Pridopidine, 4-[3-(Methylsulfonyl)phenyl]-1-propylpiperidine, is a potent Sigma-1 Receptor (SiR) agonist in clinical development for HD and ALS. More recent data, including in-vitro binding assays and in-vivo PET imaging in rats, show pridopidine acts primarily via the sigma-1 receptor (SiR). Pridopidine demonstrates a binding affinity between 100 to 500-fold higher for SiR as compared to the dopamine D2 receptor [Sahlholm K, Arhem P, Fuxe K, et al. The dopamine stabilizers ACR16 and (−) OSU6162 display nanomolar affinities at the sigma-1 receptor. Mol Psychiatry 2013; 18: 12-14.; Sahlholm K, Sijbesma J W, Maas B, et al. Pridopidine selectively occupies sigma-1 rather than dopamine D2 receptors at behaviorally active doses. Psychopharmacology (Berl). 2015; 232(18):3443-53]. The SiR is an endoplasmic reticulum (ER) protein located mainly at the Mitochondria-Associated Membrane (MAM), where it regulates diverse cellular processes including calcium signaling, ion-channel modulation, and the ER stress response [Hayashi T, Su T P. Sigma-1 receptor chaperones at the ER-mitochondrion interface regulate Ca(2+) signaling and cell survival. Cell 2007; 3: 596-610].

SiR activation is known to promote neuroprotection by stimulating neuronal survival, repair, and plasticity [Ryskamp D, Wu J, Geva M, et al. The sigma-1 receptor mediates the beneficial effects of pridopidine in a mouse model of Huntington disease. Neurobiol Dis. 2016; 97(Pt A):46-59.; Geva M, Kusko R, Soares H et al. Pridopidine activates neuroprotective pathways impaired in Huntington disease. Hum Med Gen 2016; 25 (18): 3975-3987]. Pridopidine demonstrates neuroprotective properties in several in-vivo and in-vitro HD models mediated by the SiR [Ryskamp D, Wu J, Geva M, et al. The sigma-1 receptor mediates the beneficial effects of pridopidine in a mouse model of Huntington disease. Neurobiol Dis. 2016; 97(Pt A):46-59.; Nguyen L, Lucke-Wold B P, Mookerjee S A et al. Role of sigma-1 receptors in neurodegenerative diseases. J Pharm Sci 2015; 127: 17-29].

Processes of synthesis of pridopidine and a pharmaceutically acceptable salt thereof are disclosed in U.S. Pat. Nos. 7,923,459, 10,047,049, and 6,903,120.

This invention is directed to processes and intermediates for the preparation of pridopidine.

### SUMMARY OF THE INVENTION

In some embodiments, this invention is directed to a compound represented by the structure of Formula I:

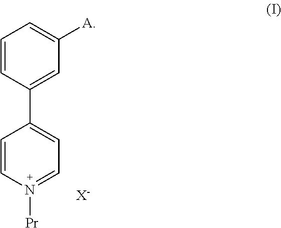

wherein A is —SMe or —SO₂Me; and
X⁻ is an anion.

In some embodiments, this invention is directed to processes for the preparation of pridopidine 4-[3-(methyl-sulfonyl)phenyl]-1-propylpiperidine):

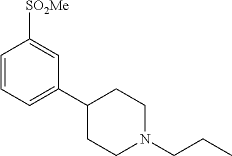 [SMILES: CCCN1CCC(c2cccc(S(C)(=O)=O)c2)CC1]

and pharmaceutically acceptable salts thereof, from a compound of Formula I.

In some embodiments, the compound of Formula I is represented by the structure of Compound 1:

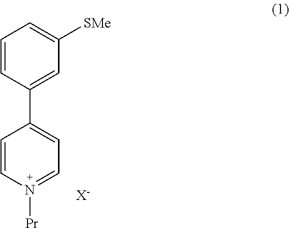

wherein X⁺ is an anion.

In some embodiments, the compound of Formula I is represented by the structure of Compound 2:

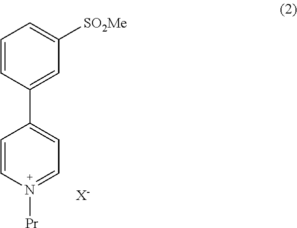

wherein X⁻ is an anion.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 presents a synthetic scheme for Process 1 for the preparation of pridopidine by a full reduction step of the pyridinium ring of intermediate of a compound of Formula I, wherein A is SMe or SO₂Me.

FIG. 2 presents a synthetic scheme of Process 2 for the preparation of pridopidine by a stepwise reduction of the pyridinium ring of intermediate of a compound of Formula I, wherein A is SMe or SO₂Me.

FIG. 3 presents a synthetic scheme of Process 3 for the preparation a compound of Formula I.

FIG. 4 presents a synthetic scheme for the preparation of pridopidine via an intermediate of a compound of Formula I, wherein A is bromide and X⁻ is an anion (Compound 3). This process is exemplified in Example 1.

FIG. 5 presents a synthetic scheme for the preparation of pridopidine by a full reduction of the pyridinium ring of an intermediate of a compound of Formula I, wherein A is SO₂Me and X⁻ is an anion (Compound 2). This process is exemplified in Example 2.

FIG. 6 presents a synthetic scheme for the preparation of pridopidine by a stepwise reduction of the pyridinium ring of an intermediate of a compound of Formula I, wherein A is SO₂Me and X⁻ is I⁻ (Compound 2). This process is exemplified in Example 3.

FIG. 7 presents a synthetic scheme for the preparation of pridopidine by a full reduction of the pyridinium ring of an intermediate of a compound of Formula I, wherein A is SMe and X⁻ is I⁻ (Compound 1). This process is exemplified in Example 4.

FIG. 8 presents a synthetic scheme for the preparation of pridopidine via an intermediate of a compound of Formula I, wherein A is SMe and X⁻ is I⁻ (Compound 1) which is oxidized to SO₂Me, wherein A is SO₂Me and X⁻ is ⁻OH (Compound 2) followed by full reduction of the pyridinium ring. This process is exemplified in Example 5.

FIG. 9 presents a synthetic scheme for the preparation of pridopidine by a stepwise reduction of the pyridinium ring of an intermediate of a compound of Formula I, wherein A is SMe (Compound 1) which is oxidized to SO₂Me followed by reduction of the tetrahydropyridine ring. This process is exemplified in Example 6.

FIG. 10 presents a synthetic scheme for the preparation of pridopidine via an intermediate of a compound of Formula I, wherein A is nitro. This process is exemplified in Example 7.

FIG. 11 presents a synthetic scheme for the preparation of pridopidine via an intermediate of a compound of Formula I, wherein A is a protected amine. This process is exemplified in Example 8.

FIG. 12 presents a synthetic scheme for the preparation of pridopidine via an intermediate of Compound 9. This process is exemplified in Example 9.

FIG. 13 presents a synthetic scheme for the preparation of pridopidine via an intermediate of Compound 10. This process is exemplified in Example 10.

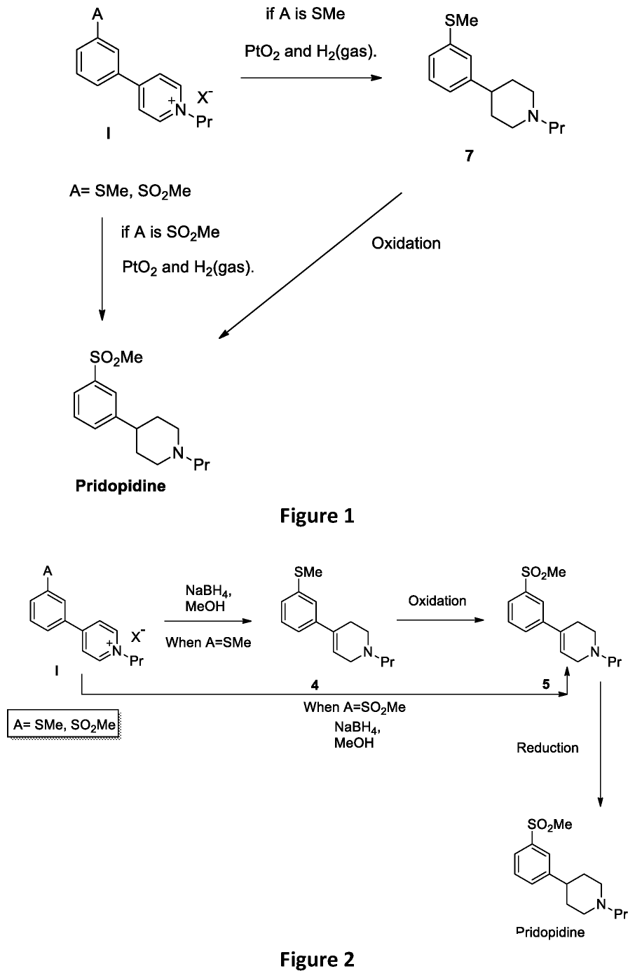

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In some embodiments, this invention is directed to a process for the preparation of pridopidine 4-[3-(Methylsulfonyl)phenyl]-1-propylpiperidine:

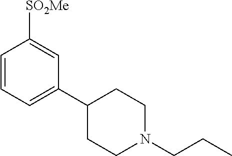 [SMILES: CCCN1CCC(c2cccc(S(C)(=O)=O)c2)CC1]

and pharmaceutically acceptable salt thereof, wherein the process comprises the use of a compound of Formula I as an intermediate:

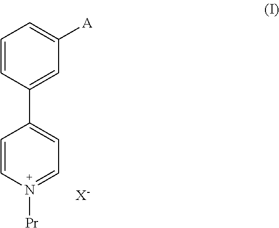

wherein
A is halide, nitro, protected amine, —SMe or —SO₂Me; and
X⁻ is an anion.

In some embodiments, this invention is directed to a process for the preparation of pridopidine 4-[3-(Methylsulfonyl)phenyl]-1-propylpiperidine:

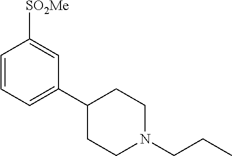 [SMILES: CCCN1CCC(c2cccc(S(C)(=O)=O)c2)CC1]

and pharmaceutically acceptable salt thereof, wherein the process comprises the use of a compound of Formula IV as an intermediate:

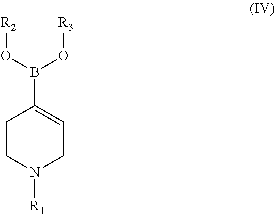

wherein
R₁ is H, propyl or a protecting group; and
R₂ and R₃ are each independently an alkyl group or R₂ and R₃ form together a 5-8 membered ring, wherein the ring is optionally substituted.

In some embodiments, this invention is directed to processes for the preparation of pridopidine 4-[3-(Methylsulfonyl)phenyl]-1-propylpiperidine:

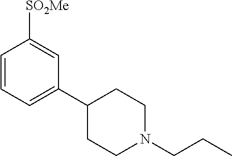 [SMILES: CCCN1CCC(c2cccc(S(C)(=O)=O)c2)CC1]

and pharmaceutically acceptable salt thereof, wherein the process comprises the use of a compound of Formula XIII as an intermediate:

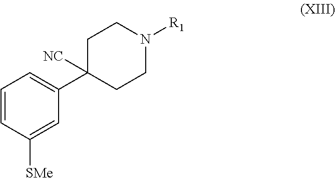

wherein
R₁ is H, propyl or a protecting group.

In one embodiment, this invention provides Process 1 for the preparation of pridopidine, the process comprises:
reducing a pyridinium group of a compound of Formula I

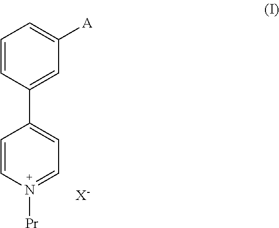

wherein
A is SMe or SO₂Me, and X⁻ is an anion to obtain respectively Compound 7:

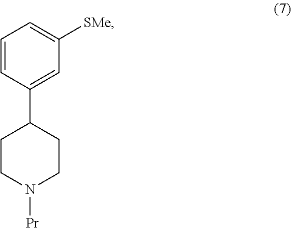

or pridopidine;
wherein, Compound 7 is further oxidized (—SMe to —SO₂Me) to obtain pridopidine.

In one embodiment, this invention provides Process 1A for the preparation of pridopidine, the process comprises:
oxidizing a compound of Formula I:

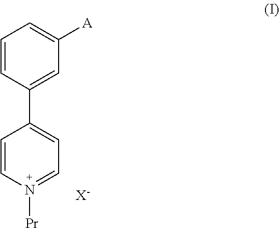

wherein,
A is SMe, and X⁻ is an anion to obtain Compound 2:

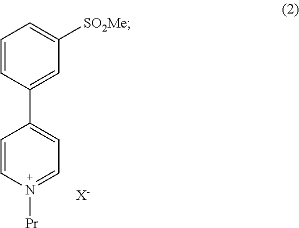

followed by reduction of the pyridinium ring to obtain pridopidine.

In one embodiment, this invention provides Process 2 for the preparation of pridopidine, the process comprises:
reducing a pyridinium ring of a compound of Formula I:

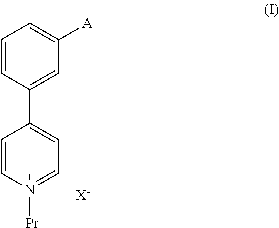

wherein A is SMe or SO₂Me, and X⁻ is an anion;
to obtain respectively Compound 4,

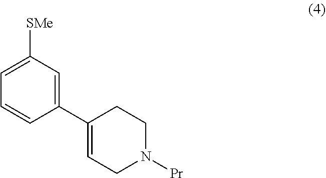

### or Compound 5

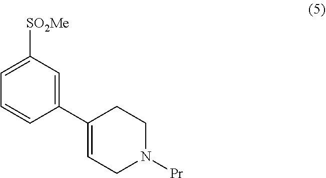

wherein, Compound 5 is further reduced (reduction of the double bond) to obtain pridopidine; or Compound 4 is further oxidized (—SMe to —SO₂Me) and then reduced (the double bond) to obtain pridopidine; or alternatively, Compound 4 is further reduced (the double bond) and then oxidized (—SMe to —SO₂Me) to obtain pridopidine.

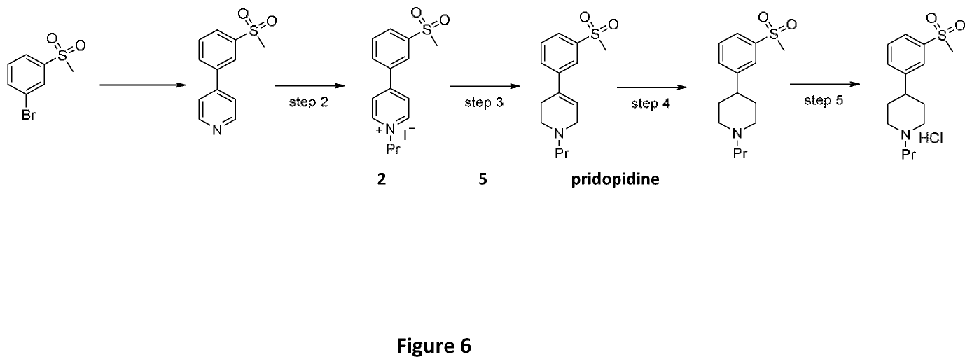

In another embodiment, pridopidine is prepared according to Example 3 and FIG. 6.

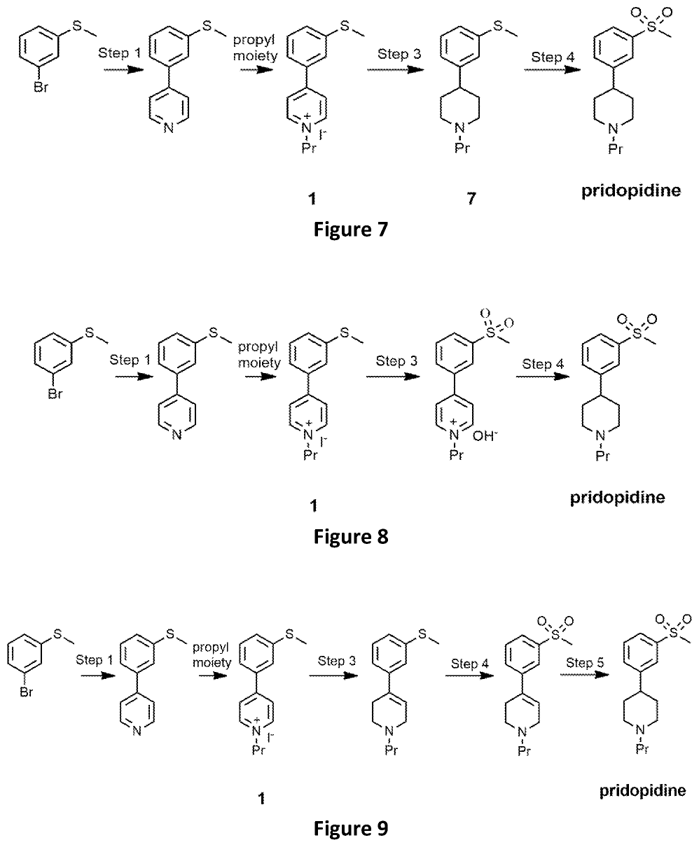

In another embodiment, pridopidine is prepared according to Example 4 and FIG. 7.

In one embodiment, this invention provides Process 3 for the preparation of a compound of Formula I:

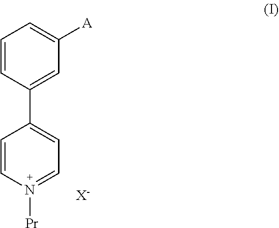

wherein A is halide, nitro, protected amine, —SMe or —SO₂Me and X⁻ is an anion; wherein the process (Process 3) comprises:
a) reacting a compound of Formula II:

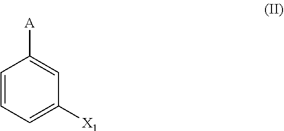

wherein A is halide, nitro, protected amine, —SMe or —SO₂Me; and X₁ is halide;

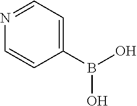

with OH (pyridin-4-ylboronic acid) in the presence of a first palladium catalyst and a weak base, to obtain a compound of Formula III

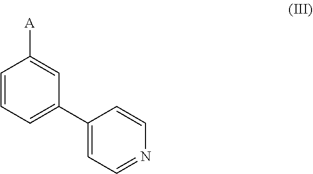

wherein A is halide, nitro, protected amine, —SMe or —SO₂Me; and
b) reacting a compound of Formula III with a propyl moiety to obtain a compound of Formula I.

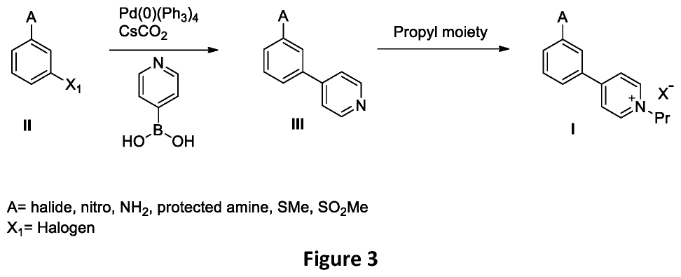

In another embodiment, a compound of Formula I is prepared as described in FIG. 3.

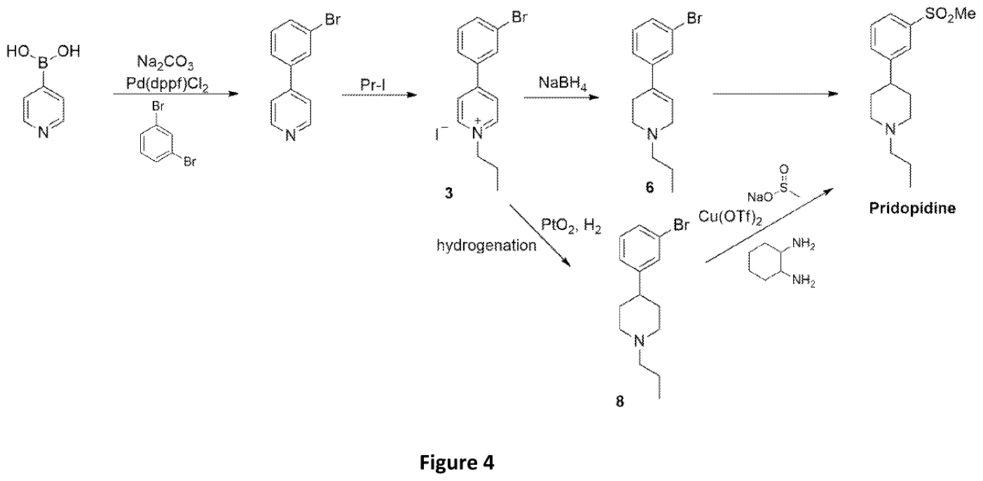

In another embodiment, a compound of Formula I is prepared as described in FIG. 4, when A is bromide.

In some embodiments, provided herein a process for the preparation of pridopidine, using a compound of Formula I as an intermediate,

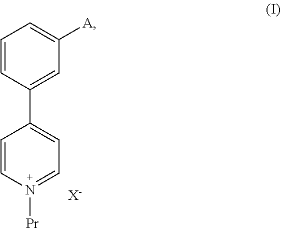

wherein A is halide, nitro, protected amine, —SMe or —SO₂Me; and X⁻ is an anion.

In one embodiment, provided herein a process for the preparation of pridopidine, using a compound of Formula I as an intermediate, wherein A is halide; and X⁻ is an anion. In another embodiment, the halide is, bromide, chloride, fluoride or iodide. In another embodiment, A is bromide. In one embodiment, provided herein a process for the preparation of pridopidine, using a compound of Formula I as an intermediate, wherein A is halide; and X⁻ is an anion, wherein the process comprises reaction between the compound of Formula I, with a SMe moiety (which will further be oxidized to SO₂Me) or with SO₂Me moiety, and further reducing the pyridinium ring to obtain pridopidine. In one embodiment, provided herein a process for the preparation of pridopidine, using a compound of Formula I as an intermediate, wherein A is halide; and X⁻ is an anion, wherein the process comprises reducing the pyridinium ring followed by reacting the halide group with SMe moiety (which will further be oxidized to SO₂Me) or with SO₂Me moiety to obtain pridopidine. In another embodiment, the oxidation of SMe to SO₂Me is performed before or after the reduction of the pyridinium ring. In another embodiment, provided herein a process for the preparation of pridopidine as presented in FIG. 4 and Example 1.

In one embodiment, provided herein a process for the preparation of pridopidine, using a compound of Formula I as an intermediate, wherein A is nitro; and X⁻ is an anion. In one embodiment, provided herein a process for the preparation of pridopidine, using a compound of Formula I as an intermediate, wherein A is nitro; and X⁻ is an anion, wherein the process comprises reducing the pyridinium ring and the nitro group to an amine followed by converting the amine to an SMe group (which will further be oxidized to SO₂Me) or to an SO₂Me group to obtain pridopidine. In another embodiment, the oxidation of SMe to SO₂Me is performed before or after the reduction of the pyridinium ring. In another embodiment, provided herein a process for the preparation of pridopidine as presented in FIG. 10 and Example 7.

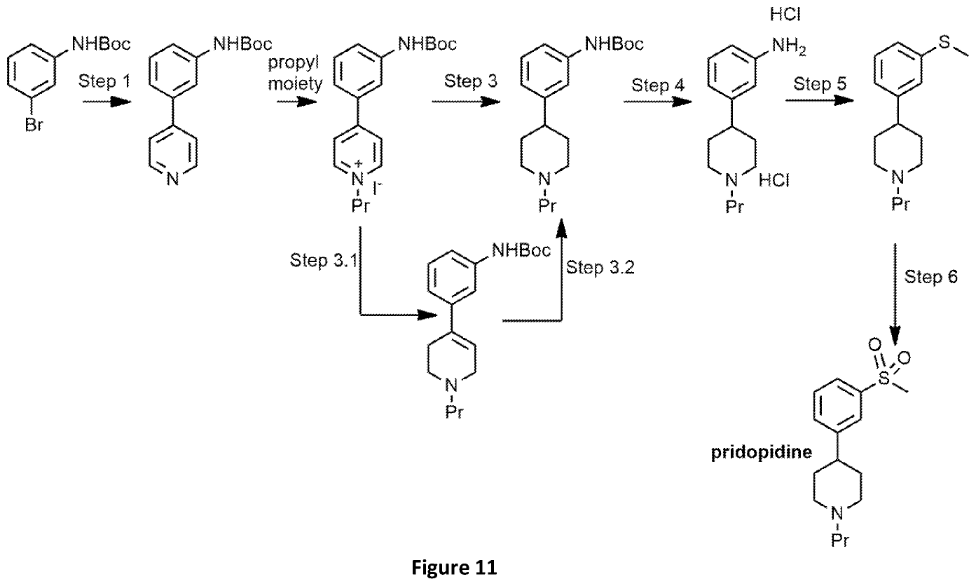

In one embodiment, provided herein a process for the preparation of pridopidine, using a compound of Formula I as an intermediate, wherein A is a protected amine; and X⁻ is an anion. In one embodiment, provided herein a process for the preparation of pridopidine, using a compound of Formula I as an intermediate, wherein A is protected amine; and X⁻ is an anion, wherein the process comprises reducing the pyridinium ring followed by removing the protecting group and converting the amine to SMe group (which will further be oxidized to SO₂Me) or to SO₂Me group to obtain pridopidine. In one embodiment, provided herein a process for the preparation of pridopidine, using a compound of Formula I as an intermediate, wherein A is a protected amine; and X⁻ is an anion, wherein the process comprises removing the protecting group and converting the amine to an SMe group (which will further be oxidized to SO₂Me) or to SO₂Me group followed by reducing the pyridinium ring to obtain pridopidine. In another embodiment, the oxidation of SMe to SO₂Me is performed before or after the reduction of the pyridinium ring. In another embodiment, the protecting group of the amine is Boc, Cbz, benzyl, Fmoc and di-benzyl. In another embodiment, provided herein a process for the preparation of pridopidine as presented in FIG. 11 and Example 8.

In one embodiment, this invention provides Process 4a for the preparation of pridopidine, wherein the process comprises:
(a) reacting the compound of Formula IV

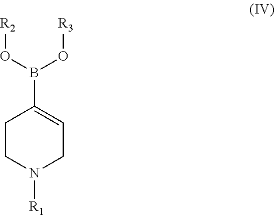

wherein R₁ is H, propyl or a protecting group; and
R₂ and R₃ are each independently an alkyl group or R₂ and R₃ form together a 5-8 membered ring, wherein the ring is optionally substituted;
   with 3-halo thioanisole in the presence of a second palladium catalyst and a weak base, to obtain a compound of Formula V:

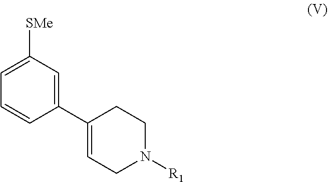

wherein if R₁ of the compound of Formula V is hydrogen (R₁=H), then, the compound is optionally reacted with a propyl moiety to obtain a derivative of Formula V (Compound 4); or if R₁ of the compound of Formula V is a protecting group (R₁=protecting group), then, the protecting group is optionally deprotected and optionally reacted with a propyl moiety to obtain a derivative of Formula V (Compound 4);
(d) oxidizing the compound of Formula V to obtain a compound of Formula VI:

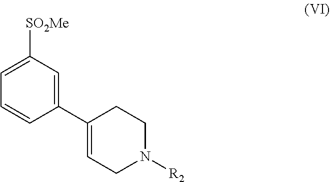

wherein
if R₁ of the compound of Formula V is hydrogen (R₁=H), then, the compound is optionally reacted with a propyl moiety to obtain the propyl derivative of Formula V (Compound 5); or if R₁ of the compound of Formula V is a protecting group (R₁=protecting group), then, the protecting group is optionally deprotected and optionally reacted with a propyl moiety to obtain the propyl derivative of Formula V (Compound 5); and
(e) reducing the compound of Formula V to obtain pridopidine or a compound of Formula VII

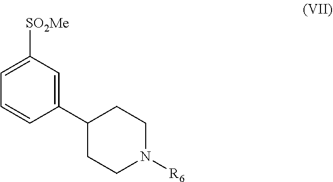

wherein R₆ is H or a protecting group;
wherein if R₆ of the compound of Formula VII is hydrogen (R₆=H), then, the compound is reacted with a propyl moiety to obtain pridopidine; or if R₆ of the compound of Formula VII is a protecting group (R₆=protecting group), then, the compound is further deprotected and reacted with propyl moiety to obtain pridopidine.

In one embodiment, this invention provides Process 4b for the preparation of pridopidine, the process comprises:
(a) reacting a compound of Formula IV:

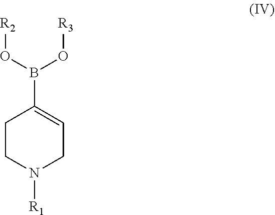

wherein
R₁ is H, a propyl or a protecting group; and
R₂ and R₃ are each independently an alkyl group or R₂ and R₃ form together a 5-8 membered ring, wherein the ring is optionally substituted;
with 1-halo-3-(methylsulfonyl)benzene, weak base and second palladium catalyst to obtain a compound of Formula VI:

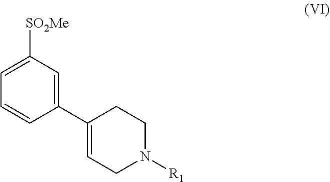

wherein if R₁ of the compound of Formula VI is hydrogen (R₁=H), then, the compound is optionally reacted with a propyl moiety to obtain the propyl derivative of Formula VI (Compound 5); or if R₁ of the compound of Formula VI is a protecting group (R₁=protecting group), then, the protecting group is optionally deprotected and optionally reacted with a propyl moiety to obtain the propyl derivative of Formula VI (Compound 5); and
(b) reducing the compound of Formula VI to obtain pridopidine or a compound of Formula VII:

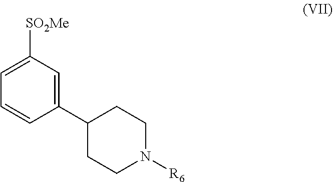

wherein R₆ is H or a protecting group;
wherein if R₆ of the compound of Formula VII is hydrogen (R₆=H), then, the compound is reacted with a propyl moiety to obtain pridopidine; or if R₆ of the compound of Formula VII is a protecting group (R₆=protecting group), then, the compound is further deprotected and reacted with propyl moiety to obtain pridopidine.

In one embodiment, this invention provides Process 4c for the preparation of pridopidine, the process comprises:
(a) reacting Compound 10

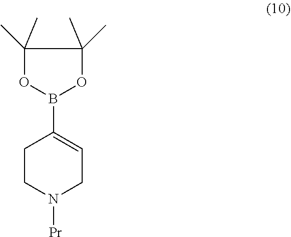

with 3-halothio anisole in the presence of a second palladium catalyst and a weak base, to obtain Compound 4:

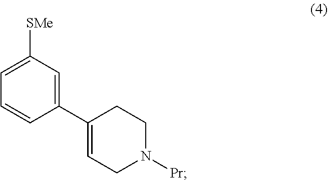

(d) oxidizing —SMe to —SO₂Me of Compound 4 to obtain Compound 5: SO₂Me

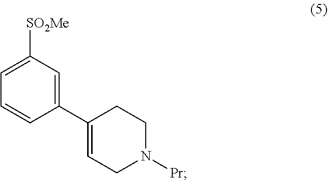

and
(e) reducing Compound 5 to obtain pridopidine.

In one embodiment, this invention provides Process 4d for the preparation of pridopidine, the process comprises:
(a) reacting Compound 10

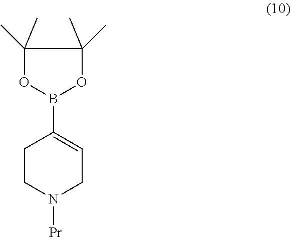

with 1-halo-3-(methylsulfonyl)benzene, weak base and second palladium catalyst to obtain Compound 5:

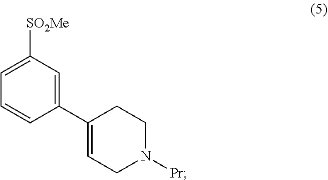

and
(b) reducing Compound 5 to obtain pridopidine.

In one embodiment, this invention provides Process 4e for the preparation of pridopidine, the process comprises:
(a) reacting the compound of Formula IV

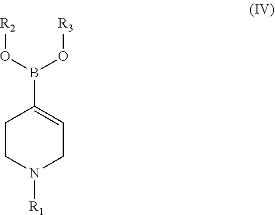

wherein R₁ is H, propyl or an amine protecting group; and R₂ and R₃ are each independently an alkyl group or R₂ and R₃ form together a 5-8 membered ring, wherein the ring is optionally substituted;
with 3-halo thioanisole in the presence of a second palladium catalyst and a weak base, to obtain a compound of Formula V:

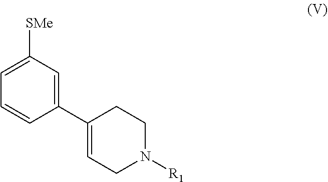

wherein if R₁ of the compound of Formula V is hydrogen (R₁=H), then, the compound is optionally reacted with a propyl moiety to obtain a derivative of Formula V (Compound 4); or if R₁ of the compound of Formula V is a protecting group (R₁=protecting group), then, the protecting group is optionally deprotected and optionally reacted with a propyl moiety to obtain a derivative of Formula V (Compound 4);
(c) reducing a compound of Formula V to obtain a compound of Formula XIV:

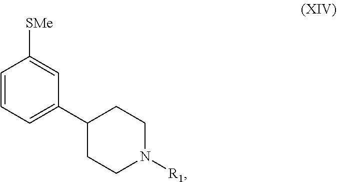

wherein if R₁ of the compound of Formula XIV is hydrogen (R₁=H), then, the compound is optionally reacted with a propyl moiety to obtain the propyl derivative of Formula XIV; or if R₁ of the compound of Formula XIV is a protecting group (R₁=protecting group), then, the protecting group is optionally deprotected and optionally reacted with a propyl moiety to obtain the propyl derivative of Formula XIV; and
(b) oxidizing the compound of Formula XIV to obtain pridopidine or a compound of Formula VII

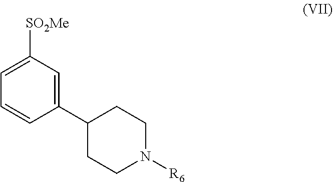

wherein R₆ is H or a protecting group;
wherein,
if R₆ of the compound of Formula VII is hydrogen (R₆=H), then, the compound is reacted with a propyl moiety to obtain pridopidine; or if R₆ of the compound of Formula VII is a protecting group (R₆=protecting group), then, the compound is further deprotected and reacted with propyl moiety to obtain pridopidine.

In one embodiment, this invention provides Process 5a for the preparation of pridopidine, the process comprises:
(a) reacting a compound of Formula IV:

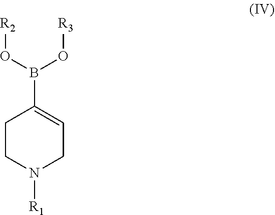

wherein
R₁ is H, a propyl or a protecting group; and
R₂ and R₃ are each independently an alkyl group or R₂ and R₃ form together a 5-8 membered ring, wherein the ring is optionally substituted;
with a compound of Formula VIII:

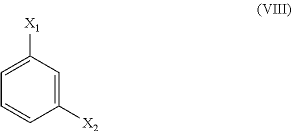

wherein
X₁ and X₂ are each independently are halo;
by Suzuki-Miyaura reaction to obtain a compound of Formula IX:

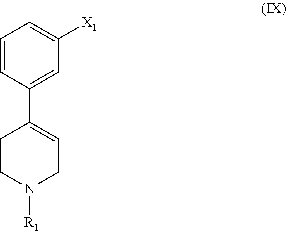

wherein
X₁ is a halo; and
R₁ is H, a propyl or a protecting group
wherein if R₁ of the compound of Formula IX is hydrogen (R₁=H), then, the compound is optionally reacted with a propyl moiety to obtain the propyl derivative of Formula IX; or if R₁ of the compound of Formula IX is a protecting group (R₁=protecting group), then, the protecting group is optionally deprotected and optionally reacted with a propyl moiety to obtain the propyl derivative of Formula IX; and
(b) Reacting compound IX wherein X₁ is a halo by an Ullmann reaction with sodium methylsulfinate and copper(II) trifluoromethanesulfonate with 1,2-diaminocyclohexane (mixture of cis and trans) as catalyst afforded the sulfone a compound of Formula VI

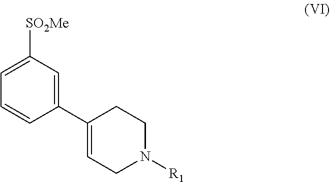

wherein if R₁ of the compound of Formula VI is hydrogen (R₁=H), then, the compound is optionally reacted with a propyl moiety to obtain the propyl derivative of Formula VI (Compound 5); or if R₁ of the compound of Formula VI is a protecting group (R₁=protecting group), then, the protecting group is optionally deprotected and optionally reacted with a propyl moiety to obtain the propyl derivative of Formula VI (Compound 5); and
(c) reducing the compound of Formula VI to obtain pridopidine or a compound of Formula VII:

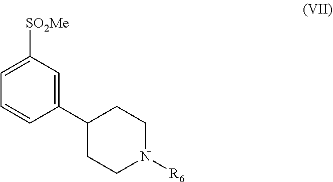

wherein R₆ is H or a protecting group;
wherein if R₆ of the compound of Formula VII is hydrogen (R₆=H), then, the compound is reacted with a propyl moiety to obtain pridopidine; or if R₆ of the compound of Formula VII is a protecting group (R₆=protecting group), then, the compound is further deprotected and reacted with propyl moiety to obtain pridopidine.

In one embodiment, this invention provides Process 5b for the preparation of pridopidine, the process comprises:
(a) reacting a Compound 10:

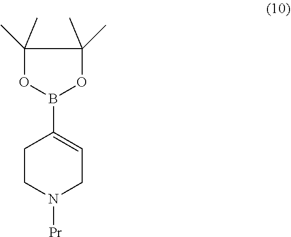

with a compound of Formula VIII:

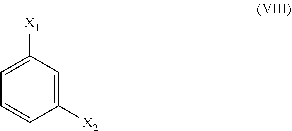

wherein
X₁ and X₂ are each independently are halo;
by Suzuki-Miyaura reaction to obtain a compound of Formula XVII:

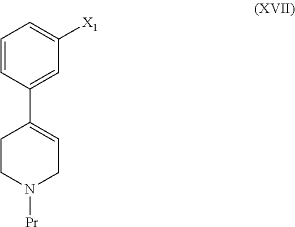

wherein
X₁ is a halo; and
(b) Reacting compound XVII wherein X₁ is a halo by an Ullmann reaction with sodium methylsulfinate and copper(II) trifluoromethanesulfonate with 1,2-diaminocyclohexane (mixture of cis and trans) as catalyst afforded the sulfone a compound of Formula V.

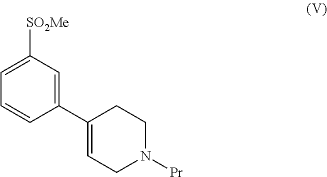

(c) reducing the compound of Formula V to obtain pridopidine.

In one embodiment, this invention provides Process 6a for the preparation of a compound of Formula IV:

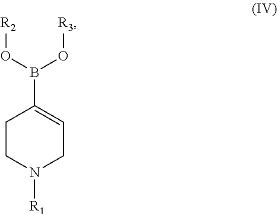

wherein, R₁ is H, a propyl or a protecting group; and
R₂ and R₃ are each independently an alkyl group or R₂ and R₃ form together a 5-8 membered ring, wherein the ring is optionally substituted;
wherein the process comprises:
(a) reacting a compound of Formula X:

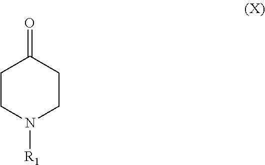

with a strong base, which is further reacted with a trifluoro moiety to obtain a compound of Formula XI:

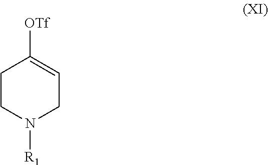

wherein if R₁ of the compound of Formula XI is hydrogen (R₁=H), then the compound is optionally reacted with a propyl moiety to obtain the propyl derivative of a compound of Formula XI; or if R₁ of the compound of Formula XI is a protecting group (R₁=protecting group), then the protecting group is optionally deprotected and optionally reacted with a propyl moiety to obtain the propyl derivative of a compound of Formula XI; and
(b) reacting the compound of Formula XI with a diboron, a moderate base and a second palladium catalyst to obtain a compound of Formula IV;

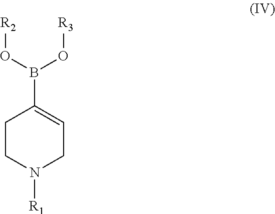

wherein if R₁ of the compound of Formula IV is hydrogen (R₁=H), the compound is optionally reacted with a propyl moiety to obtain the propyl derivative of Formula IV;
or if R₁ of the compound of Formula IV is a protecting group (R₁=protecting group), then protecting group is optionally deprotected and optionally reacted with a propyl moiety to obtain the propyl derivative of Formula IV.

In one embodiment, this invention provides Process 6b for the preparation of Compound 10:

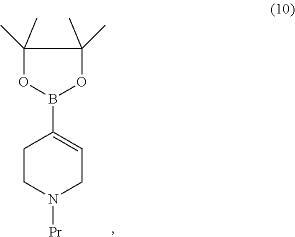

wherein the process comprises:
(b) reacting a compound of Formula X:

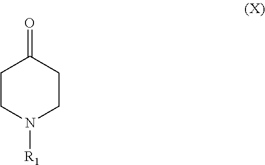

with a strong base, which is further reacted with a trifluoro moiety to obtain a compound of Formula XI:

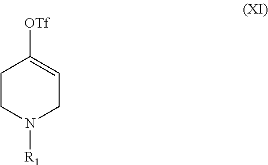

wherein if R₁ of the compound of Formula XI is hydrogen (R₁=H), then the compound is optionally reacted with a propyl moiety to obtain the propyl derivative of a compound of Formula XI; or if R₁ of the compound of Formula XI is a protecting group (R₁=protecting group), then the protecting group is optionally deprotected and optionally reacted with a propyl moiety to obtain the propyl derivative of a compound of Formula XI; and
(b) reacting the compound of Formula XI with a diboron, a moderate base and a second palladium catalyst to obtain Compound 10 or a compound of Formula XII;

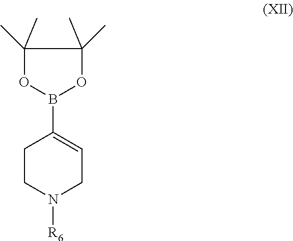

wherein if R₆ of the compound of Formula XII is hydrogen (R₁=H), then, the compound is reacted with a propyl moiety to obtain Compound 10;
or if R₆ of the compound of Formula XII is a protecting group (R₆=protecting group), then the protecting group is deprotected and reacted with a propyl moiety to obtain Compound 10.

In one embodiment, this invention provides Process 7a for the preparation of pridopidine, wherein the process comprises:
a) reacting the compound of Formula XIII

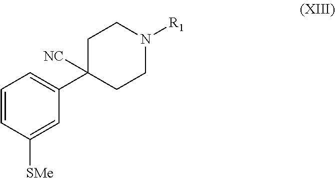

wherein
R₁ is H, propyl or a protecting group;
with a strong base, to obtain a compound of Formula XIV:

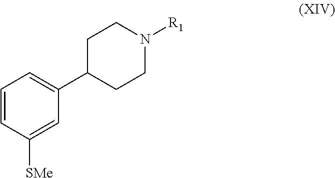

wherein if R₁ of the compound of Formula XIV is hydrogen (R₁=H), then, the compound is optionally reacted with a propyl moiety to obtain the propyl derivative of Formula XIV; or if R₁ of the compound of Formula XIV is a protecting group (R₁=protecting group), then, the protecting group is optionally deprotected and optionally reacted with a propyl moiety to obtain the propyl derivative of Formula XIV; and
(c) oxidizing the compound of Formula XIV to obtain pridopidine or a compound of Formula XV:

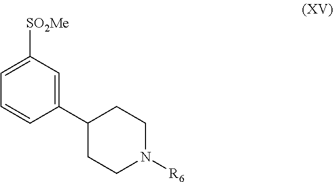

wherein R₆ is H or a protecting group;
wherein if R₆ of the compound of Formula XV is hydrogen (R₆=H), then, the compound is reacted with a propyl moiety to obtain pridopidine; or if R₆ of the compound of Formula XV is a protecting group (R₆=protecting group), then, the compound is further deprotected and reacted with propyl moiety to obtain pridopidine.

In another embodiment, this invention provides Process 7b for the preparation of pridopidine, wherein the process comprises:
(a) reacting Compound 9

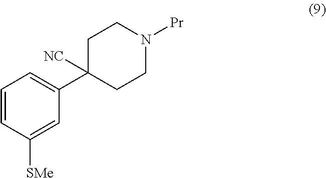

with a strong base, to obtain Compound 7:

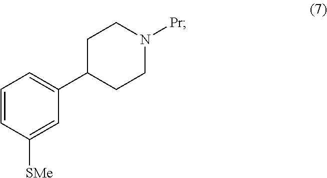

and
(c) oxidizing Compound 7 to obtain pridopidine.

In one embodiment, this invention provides Process 8a for the preparation of a compound of Formula XIII:

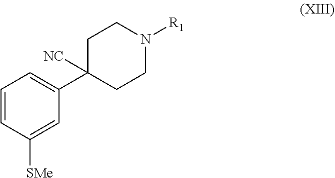

wherein R₁ is H, a propyl or a protecting group;
wherein the process comprises:
   reacting 2-(3-(methylthio)phenyl)acetonitrile with a compound of Formula XVI:

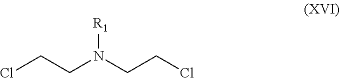

wherein R₁ is H, a propyl or a protecting group;
in the presence of a strong base, to obtain a compound of Formula XIII;
wherein if R₁ of the compound of Formula XIII is hydrogen (R₁=H), then, the compound is optionally reacted with a propyl moiety to obtain the propyl derivative of Formula XIII; or if R₁ of the compound of Formula XIII is a protecting group (R₁=protecting group), then, the protecting group is optionally deprotected and optionally reacted with a propyl moiety to obtain the propyl derivative of Formula XIII.

In one embodiment, this invention provides Process 8b for the preparation of Compound 9:

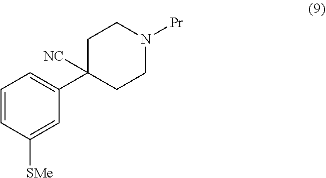

wherein the process comprises:
reacting 2-(3-(methylthio)phenyl)acetonitrile with Compound 11:

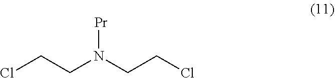

wherein Pr is propyl;
with a strong base, to obtain Compound 9.

In some embodiments provided herein a compound of Formula I and/or Compound 1 and/or Compound 2 and/or Compound 3, wherein the compound comprises an anion X⁻. In one embodiment, the anion is halide, hydroxyl, and sulfonates as mesylate, tosylate. In one embodiment, the anion is halide. In one embodiment the anion is iodide. In one embodiment, the anion is hydroxyl (OH⁻). In one embodiment, the anion is sulfonates (R—SO₂⁻).

In some embodiment provided herein a process for the preparation of pridopidine, wherein the process is via intermediate compounds of a compound of Formula I. This process includes commercially available relatively cheap (low cost) starting materials, easily isolated solid pyridinium salts, and comprises of three main steps.

In some embodiments, Process 1 comprises reduction of the pyridinium group of a compound of Formula I, wherein A is SMe or SO₂Me, to obtain Compound 7 or pridopidine, respectively. In another embodiment, the reduction step of the pyridinium ring comprises reacting a compound of Formula I with PtO₂ and H₂(gas).

In some embodiments, the processes provided herein include an oxidation step to oxidize the —SMe group to —SO₂Me group (see for example Processes 1, 2, 4a, 4c, 4e, 7a, and 7b, FIGS. 2, 7, 8, 9, 10, 11, 12). In one embodiment, the oxidation step comprises reaction with an oxidizing agent. In another embodiment, the oxidizing agent includes a tungsten catalytic oxidizing agent. In another embodiment, with the oxidizing agent includes a tungsten catalytic oxidizing agent and a peroxide.

In another embodiment, the oxidation step comprises reaction with a peroxide. In another embodiment, the oxidation step comprises reaction with a peroxide and Na₂WO₄. In another embodiment, the oxidation step comprises reaction with tungsten catalytic oxidizing agent in the presence of a peroxide oxidant at pH less than 2. In another embodiment, the oxidation step, comprises reaction with tungsten catalytic oxidizing agent in the presence of a peroxide oxidant at temperature range of 40° C. to 60° C. In another embodiment, the tungsten catalytic oxidizing agent is sodium tungstate. In another embodiment, the peroxide is sodium peroxide.

In some embodiments, the processes provided herein include a partial reduction step of a pyridinium group to obtain an alkene piperidine ring as presented below (See for example Process 2, FIGS. 2, 4, 6, 9):

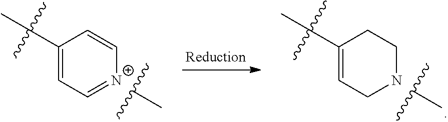

In another embodiment, the reduction step of the pyridinium group, comprises reacting a compound of Formula I with a hydrogen source. In another embodiment, the hydrogen source for the reduction of the pyridinium group is sodium borohydride, sodium cyanoborohydride, sodium triacetoxyborohydride, or H₂ (gas).

In some embodiments, the processes provided herein include a reduction step of the alkene-piperidine ring to a saturated piperidine ring (See for example Processes 2, 4a, 4b, 4c, 4d, and 4e FIGS. 2, 4, 6, 9, 13) include a reduction step for reducing the double bond to obtain the piperidine ring as presented below:

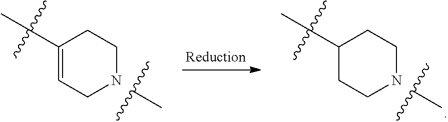

In one embodiment, the reduction step comprises a reaction with a hydrogen source for reducing the double bond to obtain the piperidine ring. In another embodiment, the reduction step for reducing the double bond to obtain the piperidine ring comprises a reaction with a hydrogen source and a catalyst. In another embodiment, the reduction step for reducing the double bond to obtain the piperidine ring is a catalytic hydrogenation in an aqueous solution with an alcohol in the presence of hydrogen source and a second palladium catalyst, a platinum catalyst or rhodium catalyst. In another embodiment, non-limiting examples of alcohol include methanol, ethanol, propanol, isopropanol and the like. In another embodiment, the third palladium catalyst is 10% palladium on carbon, 5% palladium on carbon, 5% Pd on alumina.

In another embodiment, the rhodium catalyst is 5% rhodium on carbon. In another embodiment, the platinum catalyst is platinum dioxide.

In another embodiment, the hydrogen source for the reduction step of Processes 2, 4a, 4b, 4c, 4d, and 4e or in FIGS. 2, 4, 6, 9 and 13 comprises the use of hydrogen gas, formic acid or a salt of formic acid; each is a separate embodiment according to this invention. In another embodiment, the hydrogen source of the reduction step is ammonium formate. In another embodiment, the reduction step is conducted with H₂, Pd(OH)₂/C.

In some embodiments, Process 3 comprises a reaction between a compound of Formula II and pyridine-4-ylboronic acid in the presence of first palladium catalyst to obtain a compound of Formula III. In another embodiment, the palladium catalyst comprises Tetrakis(triphenylphosphine)palladium(0) (Pd(Ph₃P)₄), Palladium(II) acetate (Pd(OAc)₂), Bis(triphenylphosphine)palladium chloride (Pd(Ph₃P)₂Cl₂), or [1,1′-Bis(diphenylphosphino)ferrocene]palladium(II) dichloride (Pd(Cl₂)dppf).

In some embodiments, Processes 3, 4a, 4b, 4c, 4d, and 4e include a step for the preparation of compounds of Formula III, V, VI, and Compounds 4 and 5 and make use of a weak base. In another embodiment, the weak base comprises potassium phosphate, sodium bicarbonate, potassium carbonate, pyridine or any combination thereof, each is a separate embodiment according to this invention.

In another embodiment, the weak base of Process 3 for the preparation of compound of Formula III, comprises sodium ethoxide and cesium carbonate or any combination thereof. In another embodiment, the weak base of Process 3 for the preparation of compound of Formula III, comprises potassium carbonate, potassium phosphate, sodium bicarbonate, sodium ethoxide and cesium carbonate or any combination thereof.

In some embodiments, the processes provided herein make use of a “propyl moiety” (See for example Processes 3, 4a, 4b, 4e, 7a, and 8a and FIGS. 7-11). In other embodiment, a “propyl moiety” refers to propionaldehyde or propyl group substituted with a leaving group. In other embodiment, a “propyl moiety” refers propyl group substituted with a leaving group. Non limiting examples of a propyl moiety include propyl bromide, propyl chloride, propyl iodide, propyl methanesulfonate, propyl p-toluenesulfonate or propyl benzene sulfonate. In some embodiments, the “propyl moiety” is propionaldehyde. In another embodiment, if the propyl moiety is propinaldehyde then the reaction is conducted by “reductive amination” and reducing agent is being add.

In another embodiment, the reducing agent is selected from hydrogen source. In another embodiment, the hydrogen source comprises NaCNBH₃, NaBH₄, NaBH(OAc)₃, H₂ (gas).

In some embodiments, the processes provided herein make use of a second palladium catalyst), for the preparation of compounds of Formula V, VI, and Compounds 4, 5 and 10, and compounds of Formula IV and XII (See for example catalyst (See for example Processes 4a, 4b, 4c, 4d, 4e 6a and 6b). In another embodiment, the second palladium catalyst is 1,1′-bis(diphenyphosphino)ferrocene-palladium dichloride, Chloro(2-dicyclohexylphosphino-2′,4′,6′-triisopropyl-1,1′-biphenyl)[2-(2′-amino-1,1′-biphenyl)]palladium(II) (Pd XPhos G2), Tetrakis(triphenylphosphine)palladium(0) (Pd(PPh₃)₄.

In some embodiments, the processes provided herein make use of a “deprotecting step”, (See for Example, Processes 4a, 4b, 5a, 6a, 6b, 7a, and 8a, or when deprotecting the protected amine in the compound of Formula I). The deprotecting step refers to deprotecting a protecting group. In one embodiment, the deprotecting step is done in acidic conditions, basic conditions or by a catalytic hydrogenation, depending on the protecting group. In another embodiment, deprotecting a Boc group is done using a trifluoroacetic acid, methanesulfonic acid, trimethylsilyl chloride or hydrochloric acid.

In another embodiment, deprotecting a Fmoc group is done using abase such as ammonia, piperidine, morpholine. In another embodiment, deprotecting a benzyl group is done by a catalytic hydrogenation. In another embodiment, the deprotection step comprises any known procedures of removal of the protecting groups and include those described in detail in Protecting Groups in Organic Synthesis, T. W. Greene and P. G. M. Wuts, 3rd edition, John Wiley & Sons, 1999 of which is incorporated entirety herein by reference.

In some embodiments, Processes 4a, 4c, and 4e include a step for the preparation of a compound of Formula V and make use of 3-halo thioanisole. In another embodiment, the 3-halo thioanisole comprises 3-bromo thioanisole, 3-chloro thioanisole, 3-iodo thioanisole; each is a separate embodiment according to this invention.

In some embodiment, Processes 6a and 6b for the preparation of a compound of Formulas IV and XII comprise the use of a moderate base. In another embodiment the moderate base comprises potassium acetate, potassium carbonate, sodium carbonate, sodium methoxide, potassium methoxide, potassium phenoxide, cesium carbonate, sodium acetate, tributyl amine, triethylamine, DBU, cesium fluoride or any combination thereof. In another embodiment the moderate base is potassium acetate.

In another embodiment, the moderate base is pyridine.

In another embodiment, the moderate base of Processes 6a and 6b for the preparation of a compound of Formulas IV and XII comprises a weak base as described herein.

In some embodiments, Processes 6a, 6b, 7a, 7b, 8a and 8b, make use of a strong base for the preparation of compounds of Formula XI, XIV XIII, and Compounds 7 and 9; each is a separate embodiment according to this invention. In one embodiment, the strong base comprises sodium hydroxide, lithium bis(trimethylsilyl)amide, potassium hydroxide, sodium amide or combination thereof, each is a separate embodiment according to this invention.

In some embodiments, Process 4b make use of 1-halo-3-(methylsulfonyl)benzene to obtain a compound of Formula VI from a compound of Formula IV. In other embodiments the 1-halo-3-(methylsulfonyl)benzene comprises 1-Bromo-3-(methylsulfonyl)benzene, 1-Chloro-3-(methylsulfonyl)benzene or 1-Iodo-3-(methylsulfonyl)benzene.

In some embodiments, Processes 6a and 6b make use of diboron for the preparation of compounds Formula IV and XII. In another embodiments, the diboron is Bis(cateholato)diboron, Bis(neopentyl glycolato)diboron, 2,2′-Bi-1,3,2,-dioxaborinane, Bis(hexenylene glycolato)diboron, Bis(diethyl-D-tartrate glycolato)diboron, Bis(N,N,N′,N′-tetramethyl-L-tartaramide glycolato)diboron or bis(pinacolato)diboron. In another embodiment, the diboron is bis(pinacolato)diboron; each is a separate embodiment according to this invention.

In some embodiments, Processes 6a and 6d make use of trifluoro moiety for the preparation of a compound of Formula XI. In another embodiments, the trifluoro moiety source is 1,1,1-trifluoro-N-phenyl-N-((trifluoromethyl)sulfonyl)methanesulfonamide, trifluoromethanelsulfonicanhydride or trimethylsilyl trifluoromethanesulfonate.

In some embodiments, R₁ of a compound of Formula IV, V, VI, IX, X, XI, XIII, XIV or XVI, is propyl; each is a separate embodiment according to this invention.

In some embodiments, R₁ of a compound of Formula IV, V, VI, IX, X, XI, XIII, XIV or XVI, is hydrogen; each is a separate embodiment according to this invention. In some embodiments, R₁ of a compound of Formula IV, V, VI, IX, X, XI, XIII, XIV or XVI, is a protecting group; each is a separate embodiment according to this invention.

In some embodiments, R₆ of a compound of Formula VII, XII or XV is a hydrogen. In another embodiments, R₆ of a compound of Formula VII, XII or XV is a protecting group.

In some embodiments, a protecting group of a compound of Formula IV, V, VI, VII, IX, X, XI, XII, XIII, XIV, XV, or XVI, comprises t-Boc (tert-butoxycarbony), Fmoc (fluorenylmethoxycarbonyl), Cbz (benzyloxycarbonyl), Bn (benzyl), Bz (benzoyl), Ts (Tosyl) or carbamate group; each is a separate embodiment according to this invention. In another embodiment, the protecting group of a compound of Formula IV, V, VI, VII, IX, X, XI, XII, XIII, XIV, XV, or XVI, is a benzyl; each is a separate embodiment according to this invention. In another embodiment, the protecting group of a compound of Formula IV, V, VI, VII, IX, X, XI, XII, XIII, XIV, XV, or XVI, is t-Boc; each is a separate embodiment according to this invention. In another embodiment, the protecting group of a compound of Formula IV, V, VI, VII, IX, X, XI, XII, XIII, XIV, XV, or XVI, is Fmoc; each is a separate embodiment according to this invention. In another embodiment, the protecting group of a compound of Formula IV, V, VI, VII, IX, X, XI, XII, XIII, XIV, XV, or XVI, is Cbz; each is a separate embodiment according to this invention. In another embodiment, the protecting group of a compound of Formula IV, V, VI, VII, IX, X, XI, XII, XIII, XIV, XV, or XVI, is Tosyl; each is a separate embodiment according to this invention. In another embodiment, the protecting group of a compound of Formula IV, V, VI, VII, IX, X, XI, XII, XIII, XIV, XV, or XVI, comprises any known amine protecting group in the art and include those described in detail in Protecting Groups in Organic Synthesis, T. W. Green and P. G. M. Wuts, 3rd edition, John Wiley & Sons, 1999 of which is incorporated entirety herein by reference.

In some embodiments, R₂ of a compound of Formula IV, is an alkyl group. In some embodiments, R₃ of a compound of Formula IV is an alkyl group. In one embodiment, R₂ and R₃, of a compound of Formula IV, form together a 5-8 membered ring, wherein the ring is optionally substituted. In another embodiment, R₂ and R₃, of compounds of Formula IV, form together a 5-8-member ring, wherein the ring comprises the O—B—O as shown below:

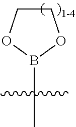

wherein the ring is optionally substituted. In another embodiment, R₂ and R₃, of a compound of Formula IV, form together substituted or unsubstituted 5-8-membered ring or form together substituted or unsubstituted fused 5-8 membered ring. In another embodiment, R₂ and R₃, of a compound of Formula IV, form together substituted or unsubstituted 5-8-membered ring, wherein the ring includes N (nitrogen), O (oxygen) or both in additional to the O—B—O atoms. In another embodiment, the N within the ring is optionally substituted with R_{A}. In another embodiment, R₂ and R₃, of a compound of Formula IV, form together substituted or unsubstituted fused 5-8-membered ring, wherein the ring includes N (nitrogen), O (oxygen) or both in addition to the O—B—O atoms. In another embodiment, the N included in the fused ring is optionally substituted with R_{A}. In another embodiment, R_{A} is alkyl, hetero alkyl, aryl, heteroaryl, alkoxy, acyl, each is a separate embodiment according to this invention. In another embodiment, the substituents comprises one or more groups such as alkyl, ester, amido, halogen, hydroxy, alkoxy, aryloxy, alkylaryloxy, heteroaryloxy, oxo, cycloalkyl, phenyl, heteroaryls, heterocyclyl, naphthyl, amino, alkylamino, arylamino, heteroarylamino, dialkylamino, diarylamino, alkylarylamino, alkylheteroarylamino, arylheteroarylamino, acyl, acyloxy, nitro, carboxy, carbamoyl, carboxamide, cyano, sulfonyl, sulfonylamino, sulfinyl, sulfinylamino, thiol, alkylthio, arylthio, or alkylsulfonyl groups, each is a separate embodiment according to this invention. Any substituents can be unsubstituted or further substituted with any one of these aforementioned substituents.

In another embodiment, R₂ and R₃, of a compound of Formula IV, form together a 5-member ring substituted with 1-4 methyl groups. In another embodiment, R₂ and R₃, of a compound of Formula IV form together a pinacol boronic ester. Non limiting examples of the R₂ and R₃ forming a ring comprising the O—B—O are presented below:

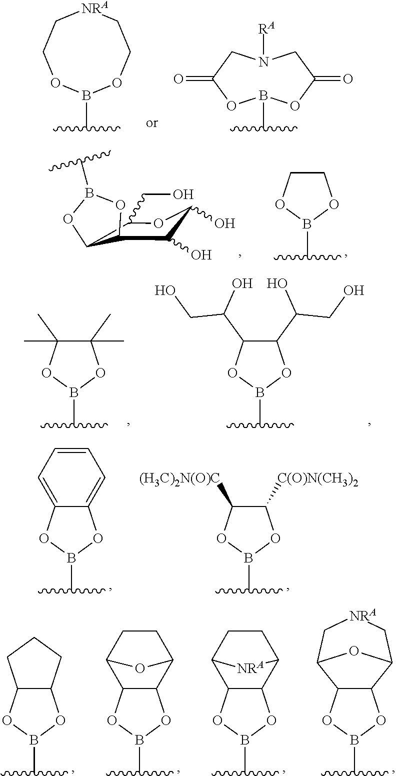

In some embodiments, X₁ of a compound of Formula II is a halide. In another embodiments, X₁ of a compound of Formula II is Br. In another embodiments, X₁ of a compound of Formula II is I. In another embodiments, X₁ of a compound of Formula II is Cl. In another embodiments, X₁ of a compound of Formula II is F.

In some embodiments, X₁ of a compound of Formula VIII, IX or XVII is a halide. In another embodiments, X₁ of a compound of Formula VIII, IX or XVII is Br. In another embodiments, X₁ of a compound of Formula VIII, IX or XVII is I. In another embodiments, X₁ of a compound of Formula VIII, IX or XVII is Cl. In another embodiments, X₁ of a compound of Formula VIII, IX or XVII is F.

In some embodiments, X₂ of a compound of Formula VIII is a halide. In another embodiments, X₂ of a compound of Formula VIII is Br. In another embodiments, X₂ of a compound of Formula VIII is I. In another embodiments, X₂ of a compound of Formula VIII is Cl. In another embodiments, X₂ of a compound of Formula VIII is F.

As used herein, the term “alkyl” used herein alone or as part of another group denotes a linear- or branched-chain alkyl group containing up to about 24 carbons unless otherwise specified. In one embodiment, an alkyl includes C₁-C₃ carbons. In one embodiment, an alkyl includes C₁-C₄ carbons. In one embodiment, an alkyl includes C₁-C₅ carbons. In another embodiment, an alkyl includes C₁-C₆ carbons. In another embodiment, an alkyl includes C₁-C₅ carbons. In another embodiment, an alkyl includes C₁-C₁₀ carbons. In another embodiment, an alkyl includes C₁-C₁₂ carbons. In another embodiment, branched alkyl is an alkyl substituted by alkyl side chains of 1 to 5 carbons. In one embodiment, the alkyl group may be unsubstituted. In another embodiment, the alkyl group may be substituted by a halogen, haloalkyl, hydroxyl, alkoxy, carbonyl, amido, alkylamido, dialkylamido, cyano, nitro, CO₂H, amino, alkylamino, dialkylamino, carboxyl, thio and/or thioalkyl.

The term “aryl” used herein alone or as part of another group denotes an aromatic ring system containing from 5-14 ring carbon atoms. The aryl ring can be a monocyclic, bicyclic, tricyclic and the like. Non-limiting examples of aryl groups are phenyl, naphthyl including 1-naphthyl and 2-naphthyl, and the like. The aryl group can be unsubstituted or substituted through available carbon atoms with one or more groups such as halogen, hydroxy, alkoxy, aryloxy, alkylaryloxy, heteroaryloxy, oxo, cycloalkyl, phenyl, heteroaryls, heterocyclyl, naphthyl, amino, alkylamino, arylamino, heteroarylamino, dialkylamino, diarylamino, alkylarylamino, alkylheteroarylamino, arylheteroarylamino, acyl, acyloxy, nitro, carboxy, carbamoyl, carboxamide, cyano, sulfonyl, sulfonylamino, sulfinyl, sulfinylamino, thiol, alkylthio, arylthio, or alkylsulfonyl groups. Any substituents can be unsubstituted or further substituted with any one of these aforementioned substituents.

The term “protecting group” refers to an amine protecting group. In another embodiment, an amine protecting group refers to any known amine protecting group such as Boc, Fmoc, Cbz,

The term “SMe moiety” refers to a SMe alkali-salt such as Me-S—Na, Me-S—K, Me-S—Li.

The term “SO₂Me moiety” refers to SO₂-alkali salt such as S(O)(ONa)Me, S(O)(OLi)Me, S(O)(OK)Me. In some embodiments, a Cu catalyst is required via Ullman reaction to convert Ph-Br to Ph-SO₂Me.

The term “pharmaceutically acceptable salt” refers to salt selected from the group consisting of hydrochloride, hydrobromide, hydroiodide, nitrate, perchlorate, phosphate, acid-phosphate, sulphate, bisulfate, formate, gluconate, glucaronate, saccharate, isonicotinate, acetate, aconate, ascorbate, benzenesulphonate, benzoate, cinnamate, citrate, embonate, enantate, fumarate, glutamate, glycolate, lactate, maleate, gentisinate, malonate, mandelate, methanesulfonate, ethanesulfonate, naphthalene-2-sulphonate, phthalate, salicylate, sorbate, stearate, succinate, tartrate, pantothenate, bitartrate, and toluene-p-sulfonate and pamoate (i.e., 1,1′-methylene-bis-(2-hydroxy-3-naphthoate) salt. Each represents a separate embodiment of this invention. In another embodiment, the pridopidine is in the form of HCl.

In one embodiment, this invention provides a compound of Formula I:

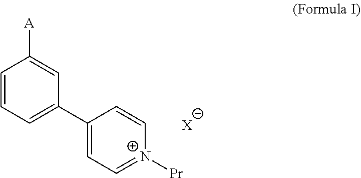

wherein A is halide, nitro, protected amine, SO₂Me or SMe, and X⁻ is an anion.

In one embodiment, this invention provides a compound of Formula I:

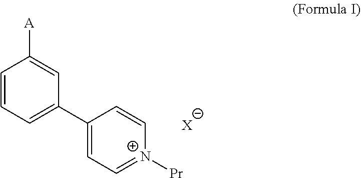

wherein A is SO₂Me or SMe.

In one embodiment, this invention provides Compound 1:

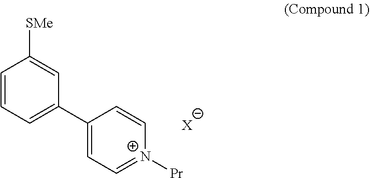

wherein X⁻ is an anion.

In one embodiment, this invention provides Compound 2:

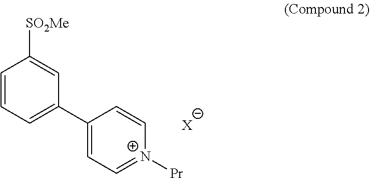

wherein X⁻ is an anion.

In one embodiment, this invention provides Compound 3:

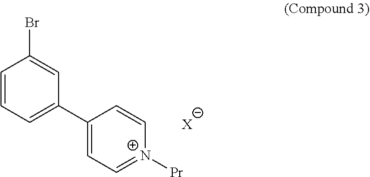

wherein X⁻ is an anion.

In one embodiment, this invention provides Compound of Formula IV:

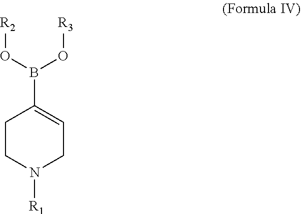

wherein
R₁ is H, propyl or a protecting group; and
R₂ and R₃ are each independently an alkyl group or R₂ and R₃ form together a 5-8 membered ring,
wherein the ring is optionally substituted.

In one embodiment, this invention provides Compound 10:

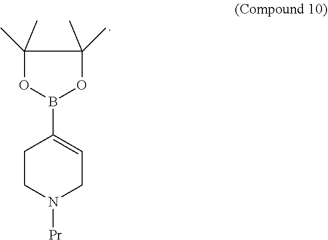

In one embodiment, this invention provides a compound of Formula XIII:

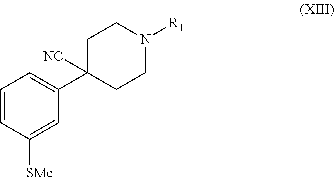

wherein
R₁ is H, propyl or a protecting group.

In one embodiment, this invention provides Compound 9:

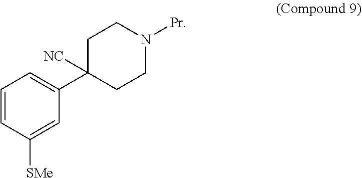

In some embodiments, this invention provides a process for the preparation of pridopidine using at least one of compounds 1, 2, 9, 10, I, IV, or XIII.

The following non-limiting examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1: Process for the Preparation of Pridopidine

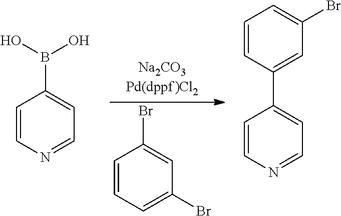

### 4-(3-Bromophenyl)pyridine

A clear solution of pyridin-4-ylboronic acid (5.00 g, 40.7 mmol), 1,3-dibromobenzene (14.4 g, 7.37 mL, 1.5 Eq, 61.0 mmol), PdCl₂(dppf) (1.49 g, 0.05 Eq, 2.03 mmol) and Na₂CO₃ (12.9 g, 61.0 mL, 2 molar, 3 Eq, 122 mmol) in 1,2-dimethoxyethane (125 mL) was degassed for 15 minutes with N₂ and afterwards heated to 85° C. for 3 hours. EtOAc (400 mL) and water (200 mL) were added. The reaction mixture was filtered over a layer of Celite and the phases were separated. The organic phase was washed with water (2×200 mL). The organic phase was dried over Na₂SO₄ and concentrated in vacuo. The crude was purified by the means of column chromatography (silica; Heptanes/EtOAc): Fraction 1 (tubes 67-95) was concentrated in vacuo to afford 4-(3-bromophenyl)pyridine (5.31 g, 22.7 mmol, 55.8%).

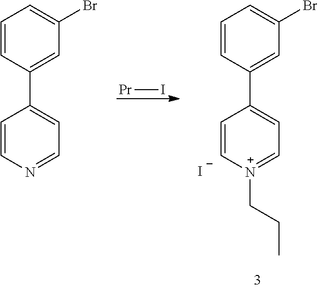

### 4-(3-Bromophenyl)-1-propylpyridin-1-ium iodide

1-Iodopropane (290 mg, 167 μL, 2.50 Eq, 1.71 mmol) was added to a solution of 4-(3-bromophenyl)pyridine (160 mg, 683 μmol) in acetonitrile (3 mL). The reaction mixture was heated to 85° C. for overnight. The volatiles were removed in vacuo to afford 4-(3-bromophenyl)-1-propylpyridin-1-ium iodide (260 mg, 643 μmol, Yield=quantitative).

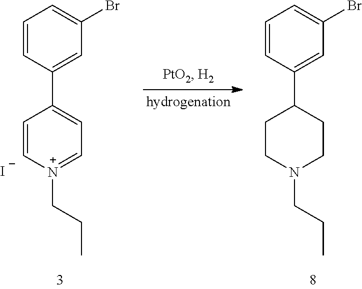 [SMILES: [I-] ; CCC[n+]1ccc(-c2cccc(Br)c2)cc1 ; CCCN1CCC(c2cccc(Br)c2)CC1]

### 4-(3-Bromophenyl)-1-propylpiperidine

PtO₂ (33.2 mg, 0.17 Eq, 146 μmol) was added to a clear brown solution of 4-(3-bromophenyl)-1-propylpyridin-1-ium iodide (348 mg, 861 μmol) in MeOH (5 mL). The reaction mixture was stirred under 5 bar hydrogen pressure at room temperature for 2 nights. The material was diluted with MeOH and filtered over a layer of Celite. The filter cake was washed with MeOH. The combined filtrates were concentrated in vacuo to afford 276 mg (I salt). The material was dissolved in EtOAc (20 mL) and sat. aq. NaHCO₃ (20 mL). The layers were separated and the organic layer was washed with sat. aq. NaHCO₃ (20 mL). The combined aqueous phases were extracted with EtOAc (20 mL). The combined organic phases were dried over Na₂SO₄ and concentrated in vacuo to afford 190 mg (free base). The material was purified by the means of column chromatography (silica; 0-5% 7N NH₃ in MeOH in DCM): Fraction 1 (tubes 8-25) was concentrated to afford 4-(3-bromophenyl)-1-propylpiperidine (164 mg, 581 μmol, Yield=67.5%).

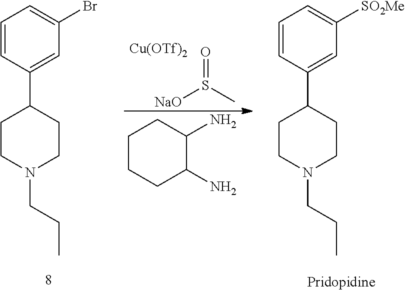

### Pridopidine via 4-(3-bromophenyl)-1-propylpiperidine (8)

A mixture of 4-(3-bromophenyl)-1-propylpiperidine (164 mg, 581 μmol), sodium methanesulfinate (89.0 mg, 1.5 Eq, 872 μmol), copper(II)trifluoromethanesulfonate (21.0 mg, 0.1 Eq, 58.1 μmol) and 1,2-diaminocyclohexane (mixture of cis and trans) (26.5 mg, 28.3 μL, 0.4 Eq, 232 μmol) in DMSO (3 mL) was deoxygenated and purged with nitrogen and then stirred at 190° C. overnight for 6 hours. EtOAc (50 mL) and water (50 mL) were added. The layers were separated. The organic phase was washed with water (50 mL), dried over Na₂SO₄ and concentrated in vacuo. The crude was purified by the means of column chromatography (silica; 0-10% MeOH in DCM): Fraction 1 (tubes 15-20) was concentrated in vacuo to afford 4-(3-(methylsulfonyl)phenyl)-1-propylpiperidine (25 mg, 89 μmol, Yield=15%).

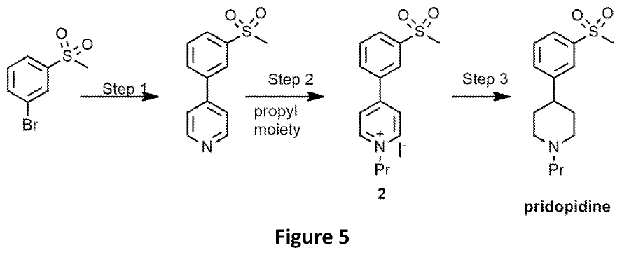

### Example 2: Process for the Preparation of Pridopidine [FIG. 5]

### Step 1: Suzuki Coupling

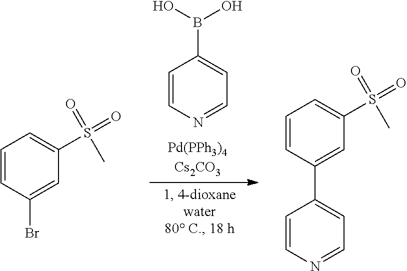 [SMILES: CS(=O)(=O)c1cccc(Br)c1 ; OB(O)c1ccncc1 ; CS(=O)(=O)c1cccc(-c2ccncc2)c1]

To a solution of 3-bromophenyl methyl sulfone (3.0 g, 12.76 mmol, 1.0 eq) in 1,4-dioxane (60 mL) and water (6 mL), pyridine-4-boronic acid (1.88 g, 15.31 mmol, 1.2 eq) and Cs₂CO₃ (12.47 g, 38.28 mmol, 3.0 eq) were added and reaction mixture was purged with argon for 15 minutes. Pd(PPh₃)₄ (740 mg, 0.64 mmol, 0.05 eq) was added and the mixture was stirred at 80° C. overnight. After this time, the mixture was cooled to room temperature, filtered through the pad of Celite®, washed with DCM and concentrated under reduced pressure. The residue was taken up in DCM, washed with water (3×), organic layer was dried over sodium sulphate, filtered and concentrated under reduced pressure. Crude material was purified by silica column chromatography (DCM:acetone 100:0->80:20) to give 3.03 g (Yield=quantitative) of 4-(3-methanesulfonylphenyl)pyridine as a brown oil with 99% of LCMS purity. LCMS (ESI): exact mass for C12H11NO2S: 233.29; [M+H]⁺=233.60 found.

¹H NMR (300 MHz, DMSO-d₆) δ 8.72 (d, J=1.6 Hz, 2H), 8.29 (t, J=1.6 Hz, 1H), 8.17 (d, J=7.9 Hz, 1H), 8.03 (d, J=8.0 Hz, 1H), 7.87-7.75 (m, 3H), 7.67-7.50 (m, 2H), 3.33 (s, 3H).

### Step 2: N-propylation

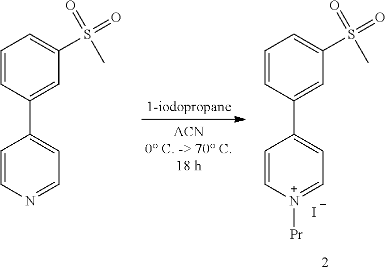

4-(3-Methanesulfonylphenyl)pyridine (560 mg, 2.43 mmol, 1.0 eq) was dissolved in ACN (10 mL) and placed in an ice-cooled bath. 1-Iodopropane (474 μL, 4.86 mmol, 2.0 eq) was added dropwise and the reaction mixture was heated to 70° C. overnight. Upon cooling to room temperature, the reaction mixture was quenched with water, and extracted with EtOAc (×3). Combined organic layers were dried over anhydrous Na₂SO₄, filtered and concentrated under reduced pressure. Obtained solid was refluxed with EtOAc (10 mL) for 1h, cooled down to room temperature, washed with EtOAc and dried under reduced pressure to give 800 mg (Yield=81%) of 4-(3-methanesulfonylphenyl)-1-propylpyridine iodide as a brown solid with 100% of LCMS purity. LCMS (ESI): exact mass for C15H18NO2S: 276.37; [M+H]⁺=276.60 found.

¹H NMR (300 MHz, DMSO-d₆) δ 9.21 (d, J=6.9 Hz, 2H), 8.66 (d, J=6.9 Hz, 2H), 8.54 (s, 1H), 8.42 (d, J=8.0 Hz, 1H), 8.18 (d, J=8.0 Hz, 1H), 7.93 (t, J=7.9 Hz, 1H), 4.60 (t, J=7.2 Hz, 2H), 3.36 (s, 3H), 1.98 (h, J=7.3 Hz, 2H), 0.92 (t, J=7.4 Hz, 3H).

### Step 3: Hydrogenation

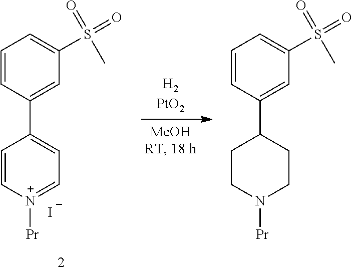

To a solution of 4-(3-methanesulfonylphenyl)-1-propylpyridine iodide (800 mg, 1.98 mmol, 1.0 eq) in methanol (20.0 mL) PtO₂ (90 mg, 0.4 mmol, 0.2 eq) was added under gentle flow of argon and the reaction mixture was evacuated and backfilled with hydrogen three times. Afterwards it was stirred under hydrogen atmosphere (balloon) overnight. Upon reaching completion, the mixture was filtered through the pad of Celite® and solvent was removed under reduced pressure. DCM (10 mL) and 2M NaOH solution (10 mL) were added and the mixture was stirred for 30 minutes at RT. Phases were separated and the water phase wase washed with DCM (2×). Organic phases were combined, dried over sodium sulphate, filtered and evaporated to give 635 mg (Yield=quantitative) of 4-(3-(methylsulfonyl)phenyl)-1-propylpiperidine with 100% of LCMS purity which was purified in the next step during the HCl salt formation. LCMS (ESI): exact mass for C15H23NO2S: 281.; [M+H]⁺=281.65 found.

### Step 4: HCl Salt Formation

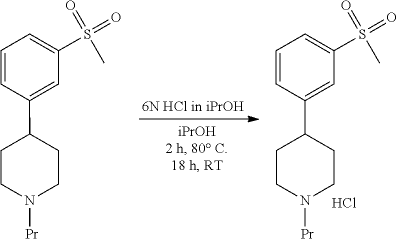

To a solution of 4-(3-(methylsulfonyl)phenyl)-1-propylpiperidine (558 mg, 1.98 mmol, 1.0 eq) was dissolved in iPrOH (6.0 mL), then 6N HCl in iPrOH was added dropwise (400 μl, 2.38 mmol, 1.2 eq) The reaction mixture was stirred 2 h at 80° C. and at room temperature overnight. The precipitate was filtered off, washed with iPrOH and dried under reduced pressure to give 492 mg (Yield=88%) of 4-(3-methanesulfonylphenyl)pyridine hydrochloride as a pale yellow solid with 100% of LCMS purity. LCMS (ESI): exact mass for C15H23NO2S: 281.14; [M+H]⁺=281.65 found. HPLC purity: 99.89% (@268 nm)

¹H NMR (300 MHz, Methanol-d₄) δ 7.88-7.79 (m, 2H), 7.69-7.55 (m, 2H), 3.74-3.63 (m, 2H), 3.20-2.98 (m, 8H), 2.24-1.94 (m, 4H), 1.90-1.71 (m, 2H), 1.02 (t, J=7.4 Hz, 3H).

### Example 3: Process for the Preparation of Pridopidine [FIG. 6]

Compound 2 was prepared as described in Example 2.

Reduction with Sodium Borohydride

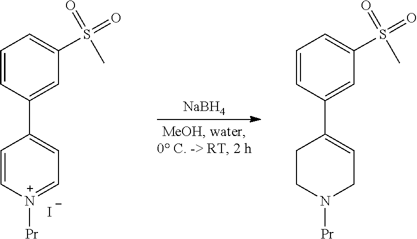

4-(3-methanesulfonylphenyl)-1-propylpyridine iodide (1.0 g, 3.62 mmol, 1.0 eq) was dissolved in methanol (10.0 mL) and water (20.0 mL) and the solution was placed in an ice-cooled bath. NaBH₄ (821 mg, 21.71 mmol, 6 eq) was added and reaction mixture was left for 2 h at ambient temperature. After that, the mixture was quenched with 1N solution of HCl and extracted with DCM (×3). Organic phases were combined, dried over sodium sulphate, filtered and evaporated to give 726 mg (Yield=66%) of 4-(3-methanesulfonylphenyl)-1-propyl-1,2,3,6-tetrahydropyridine with 93% of LCMS purity which was used as is in the next step. LCMS (ESI): exact mass for C15H21NO2S: 279.40; [M+H]⁺=279.60 found.

¹H NMR (300 MHz, DMSO-d₆) δ 7.92-7.89 (m, 1H), 7.80 (d, J=1.7 Hz, 1H), 7.77 (s, 1H), 7.61 (t, 1H), 6.37-6.30 (m, 1H), 3.23 (s, 3H), 3.09 (q, J=3.0 Hz, 2H), 2.69-2.58 (m, 2H), 2.56-2.51 (m, 2H), 2.41-2.30 (m, 2H), 1.58-1.43 (m, 2H), 0.88 (t, J=7.4 Hz, 3H).

### Step 4. Hydrogenation Reaction

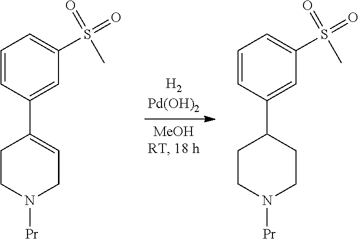

To a solution of 4-(3-methanesulfonylphenyl)-1-propyl-1,2,3,6-tetrahydropyridine (616 mg, 2.20 mmol, 1.0 eq) in methanol (30.0 mL) Pd(OH)₂/C (20% wt. loading, 50% wet, 62 mg, 0.4 mmol, 0.2 eq) was added under gentle flow of argon and the reaction mixture was evacuated and backfilled with hydrogen three times. Afterwards it was stirred under hydrogen atmosphere (balloon) overnight. Upon completion, the mixture was filtered through the pad of Celite® and solvent was removed under reduced pressure. DCM (10 mL) and 2M NaOH solution (10 mL) were added and the mixture was stirred for 30 minutes at room temperature. Phases were separated and the water phase was extracted with DCM (2×). Organic phases were combined, dried over sodium sulphate, filtered and evaporated to give 548 mg (Yield=88%) of 4-(3-(methylsulfonyl)phenyl)-1-propylpiperidine with 100% of LCMS purity which was purified in the next step during the HCl salt formation. LCMS (ESI): exact mass for C15H23NO2S: 281.14; [M+H]⁺=281.65 found.

¹H NMR (300 MHz, DMSO-d₆) δ 7.83-7.70 (m, 2H), 7.67-7.52 (m, 2H), 3.21 (s, 3H), 3.02-2.90 (m, 2H), 2.71-2.54 (m, 1H), 2.30-2.20 (m, 2H), 2.02-1.91 (m, 2H), 1.83-1.72 (m, 2H), 1.71-1.60 (m, 2H), 1.54-1.37 (m, 2H), 0.87 (t, J=7.4 Hz, 3H).

### Step 5: HCl Salt Formation

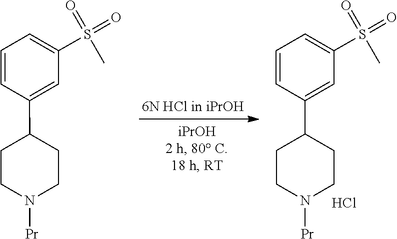

To a solution of 4-(3-(methylsulfonyl)phenyl)-1-propylpiperidine (548 mg, 1.94 mmol, 1.0 eq) was dissolved in iPrOH (6.0 mL), then 6N HCl in iPrOH was added dropwise (653 μl, 3.89 mmol, 2 eq) The reaction mixture was stirred 2 h at 80° C. and at room temperature overnight. The precipitate was filtered off, washed with iPrOH and dried under reduced pressure to give 170 mg (Yield=30%) of 4-(3-methanesulfonylphenyl)pyridine hydrochloride as a pale yellow solid with 100% of LCMS purity. LCMS (ESI): exact mass for C15H23NO2S: 281.14; [M+H]⁺=281.65 found.

HPLC purity: 99.89% (@268 nm).

¹H NMR (300 MHz, Methanol-d₄) δ 7.95-7.81 (m, 2H), 7.76-7.56 (m, 2H), 3.80-3.61 (m, 2H), 3.25-3.01 (m, 8H), 2.29-1.98 (m, 4H), 1.94-1.75 (m, 2H), 1.06 (t, J=7.4 Hz, 3H).

### Example 4: Process for the Preparation of Pridopidine [FIG. 7]

### Step 1: Suzuki Coupling

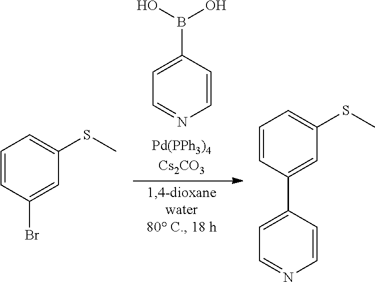

To a solution of 1-bromo-3-(methylsulfanyl)benzene (5.0 g, 24.61 mmol, 1.0 eq) in 1,4-dioxane (200 mL) and water (20 mL), pyridine-4-boronic acid (3.93 g, 32.00 mmol, 1.3 eq) and Cs₂CO₃ (24.06 g, 73.85 mmol, 3.0 eq) were added and reaction mixture was purged with argon for 15 minutes. Pd(PPh₃)₄ (2.84 g, 2.46 mmol, 0.1 eq) was added and the mixture was stirred at 80° C. overnight. After this time, the mixture was cooled to room temperature, filtered through the pad of Celite®, washed with DCM and concentrated under reduced pressure. The residue was taken up in DCM, washed with water (3×), organic layer was dried over sodium sulphate, filtered and concentrated under reduced pressure. Crude material was purified by silica column chromatography (DCM:acetone 100:0->80:20) to give 3.0 g (Yield=48%) of 4-[3-(methylsulfanyl)phenyl]pyridine as a brown oil with 96% of LCMS purity. LCMS (ESI): exact mass for C12H11NS: 201.29; [M+H]⁺=202.05 found.

¹H NMR (300 MHz, DMSO-d₆) δ 8.68-8.59 (m, 2H), 7.76-7.69 (m, 2H), 7.62 (t, J=1.7 Hz, 1H), 7.55 (d, J=7.7 Hz, 1H), 7.45 (t, J=7.7 Hz, 1H), 7.36 (d, J=7.9 Hz, 1H), 2.55 (s, 3H).

### Step 2: N-alkylation

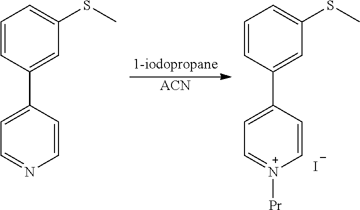

4-[3-(methylsulfanyl)phenyl]pyridine (3.0 g, 14.9 mmol, 1.0 eq) was dissolved in ACN (60 mL) and placed in an ice-cooled bath. 1-Iodopropane (2.91 mL, 29.80 mmol, 2.0 eq) was added dropwise and the reaction mixture was heated to 70° C. overnight. Upon cooling to room temperature, the reaction mixture was quenched with water, and extracted with EtOAc (×3). Combined organic layers were dried over anhydrous Na₂SO₄, filtered and concentrated under reduced pressure. Obtained solid was refluxed with EtOAc (10 mL) for 1h, cooled down to room temperature, washed with EtOAc and dried under reduced pressure to give 5.0 g (Yield=86%) of 4-[3-(methylsulfanyl)phenyl]-1-propylpyridin-1-ium iodide as a brown solid with 95% of LCMS purity. LCMS (ESI): exact mass for C15H18NS: 244.12; [M]+=243.80 found.

¹H NMR (300 MHz, DMSO-d₆) δ 9.16-9.06 (m, 2H), 8.61-8.51 (m, 2H), 7.91-7.77 (m, 2H), 7.62-7.48 (m, 2H), 4.56 (t, J=7.3 Hz, 2H), 2.59 (s, 3H), 1.98 (h, J=7.3 Hz, 2H), 0.91 (t, J=7.3 Hz, 3H).

### Step 3: Hydrogenation

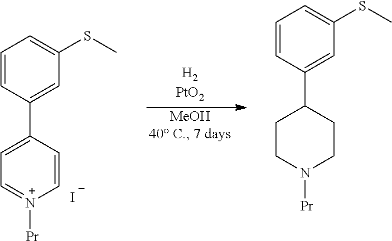

To a solution of 4-[3-(methylsulfanyl)phenyl]-1-propylpyridin-1-ium iodide (1.0 g, 4.09 mmol, 1.0 eq) in methanol (50 mL) PtO₂ (186 mg, 0.81 mmol, 0.2 eq) was added under gentle flow of argon and the reaction mixture was evacuated and backfilled with hydrogen three times. Afterwards it was stirred under hydrogen atmosphere (balloon) for 7 days at 40° C.

Upon reaching completion, the mixture was filtered through the pad of Celite® and solvent was removed under reduced pressure. DCM (20 mL) and 2M NaOH solution (20 mL) were added and the mixture was stirred for 30 minutes at RT. Phases were separated and the water phase was washed with DCM (2×). Organic phases were combined, dried over sodium sulphate, filtered and evaporated. The crude material was purified by flash column chromatography (ACN:water 40:60) to give 292 mg (Yield=25%) of 4-[3-(methylsulfanyl)phenyl]-1-propylpiperidine with 90% of LCMS purity. LCMS (ESI): exact mass for C15H23NS: 249.16.; [M+H]⁺=250.10 found.

¹H NMR (300 MHz, DMSO-d₆) δ 7.38-7.31 (m, 2H), 7.28-7.20 (m, 2H), 3.61-3.51 (m, 2H), 3.14-2.94 (m, 4H), 2.89-2.74 (m, 1H), 2.47 (s, 3H), 2.10-1.96 (m, 2H), 1.95-1.76 (m, 2H), 1.77-1.59 (m, 2H), 0.93 (t, J=7.4 Hz, 3H).

### Step 4: Oxidation

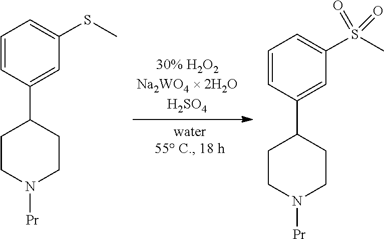

To a solution of 4-[3-(methylsulfanyl)phenyl]-1-propylpiperidine (292 mg, 1.17 mmol, 1.0 eq) in water (14.6 mL), 96% H₂SO₄ (770 μL, 14.48 mmol, 12.37 eq) was added followed by sodium tungstate dihydrate (27 mg, 0.08 mmol, 0.07 eq) and 30% H₂O₂ (90 μL, 2.92 mmol, 2.50 eq). The reaction mixture was stirred at 55° C. for 2 h, after which time it was cooled to 10° C. and toluene (50 mL) was added followed by NaOH solution. The aqueous layer was extracted with toluene (3×). Organic layers were combined, dried over sodium sulphate and evaporated under reduced pressure to give 124 mg (Yield=30%) of 4-(3-methanesulfonylphenyl)-1-propylpiperidine as a brown solid with 62% of LCMS purity which was purified in the next step during the HCl salt formation. LCMS (ESI): exact mass for C15H23NO2S: 281.14; [M+H]⁺=282.10 found.

### Step 5: HCl Salt Formation

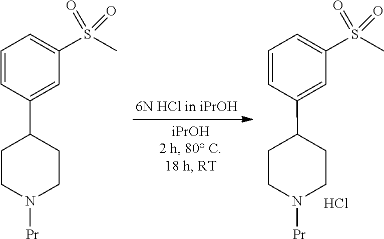

To a solution of 4-(3-(methylsulfonyl)phenyl)-1-propylpiperidine (124 mg, 0.44 mmol, 1.0 eq) was dissolved in iPrOH (3.0 mL), then 6N HCl in iPrOH was added dropwise (147 μl, 0.88 mmol, 1.2 eq) The reaction mixture was stirred 2 h at 80° C. and at room temperature overnight. The precipitate was filtered off, washed with iPrOH and dried under reduced pressure to give 13 mg (Yield=9%) of 4-(3-(methylsulfonyl)phenyl)-1-propylpiperidine hydrochloride as a pale yellow solid with 100% of LCMS purity. LCMS (ESI): exact mass for C15H23NO2S: 281.14; [M+H]⁺=281.65 found.

HPLC purity: 97.06% (@268 nm).

¹H NMR (300 MHz, Methanol-d₄) δ 7.92-7.83 (m, 2H), 7.72-7.58 (m, 2H), 3.75-3.61 (m, 2H), 3.20-3.01 (m, 8H), 2.25-1.93 (m, 4H), 1.88-1.77 (m, 2H), 1.06 (t, J=7.4 Hz, 3H).

### Example 5: Process for the Preparation of Pridopidine [FIG. 8]

Compound 1 was prepared according to Example 4 (Steps 1 and 2 in FIG. 7).

### Step 3: Oxidation

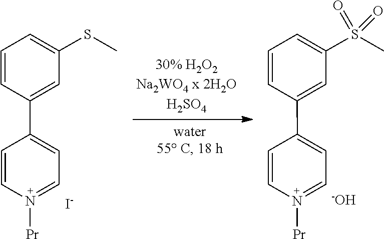

To a solution of 4-[3-(methylsulfanyl)phenyl]-1-propylpyridin-1-ium iodide (1.0 g, 4.09 mmol, 1.0 eq)) in water (50 mL), 96% H₂SO₄ (2.69 mL, 50.61 mmol, 12.37 eq) was added followed by sodium tungstate dihydrate (94 mg, 0.286 mmol, 0.07 eq) and 30% H₂O₂ (313 μL, 10.23 mmol, 2.50 eq). The reaction mixture was stirred at 55° C. for 2 h, after which time it was cooled to 10° C. and toluene (50 mL) was added followed by NaOH solution. The aqueous layer was extracted with toluene (3×). Organic layers were combined, dried over sodium sulphate and evaporated under reduced pressure to give 630 mg (Yield=50%) of 4-(3-methanesulfonylphenyl)-1-propylpyridin-1-ium hydroxide as a yellow oil with 96% of LCMS purity. LCMS (ESI): exact mass for C15H18NO2S: 276.11; [M+H]⁺=277.35 found ¹H NMR (300 MHz, DMSO-d₆) δ 9.46-9.05 (m, 2H), 8.84-7.51 (m, 6H), 4.71-4.50 (m, 2H), 3.36 (s, 3H), 2.07-1.86 (m, 2H), 1.03-0.82 (m, 3H).

### Step 4: Hydrogenation

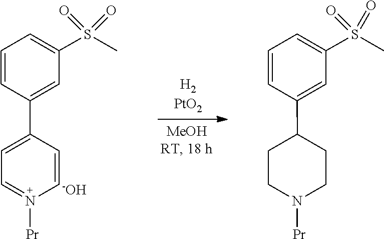

To a solution of 4-(3-methanesulfonylphenyl)-1-propylpyridin-1-ium hydroxide (630 mg, 2.15 mmol, 1.0 eq) in methanol (31 mL) PtO₂ (117 mg, 0.516 mmol, 0.2 eq) was added under gentle flow of argon and the reaction mixture was evacuated and backfilled with hydrogen three times. Afterwards it was stirred under hydrogen atmosphere (balloon) overnight. Upon reaching completion, the mixture was filtered through the pad of Celite® and solvent was removed under reduced pressure. DCM (20 mL) and 2M NaOH solution (20 mL) were added and the mixture was stirred for 30 minutes at RT. Phases were separated and the water phase was washed with DCM (2×). Organic phases were combined, dried over sodium sulphate, filtered and evaporated to give 512 mg (Yield=85%) of 4-(3-methanesulfonylphenyl)-1-propylpiperidine with 100% of LCMS purity which was purified in the next step during the HCl salt formation. LCMS (ESI): exact mass for C15H23NO2S: 281.14.; [M+H]⁺=282.10 found.

### Step 5: HCl Salt Formation

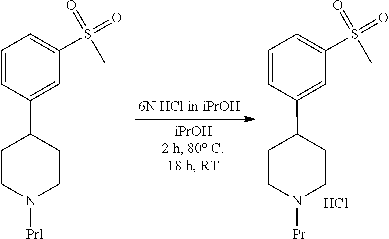

To a solution of 4-(3-(methylsulfonyl)phenyl)-1-propylpiperidine (512 mg, 1.81 mmol, 1.0 eq) was dissolved in iPrOH (5.0 mL), then 6N HCl in iPrOH was added dropwise (606 μl, 3.63 mmol, 2.0 eq) The reaction mixture was stirred 2 h at 80° C. and at room temperature overnight. The precipitate was filtered off, washed with iPrOH and dried under reduced pressure to give 50 mg (Yield=9%) of 4-(3-(methylsulfonyl)phenyl)-1-propylpiperidine hydrochloride as a pale yellow solid with 100% of LCMS purity. LCMS (ESI): exact mass for C15H23NO2S: 281.14; [M+H]⁺=281.65 found.

HPLC purity: 92.58% (@268 nm).

¹H NMR (300 MHz, Methanol-d₄) δ 7.92-7.81 (m, 2H), 7.74-7.57 (m, 2H), 3.78-3.61 (m, 2H), 3.22-3.01 (m, 8H), 2.25-1.97 (m, 4H), 1.92-1.76 (m, 2H), 1.06 (t, J=7.4 Hz, 3H).

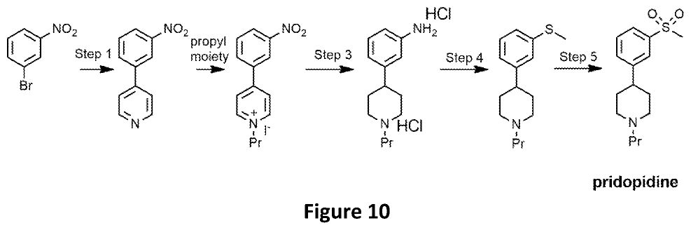

### Example 6: Process for the Preparation of Pridopidine [FIG. 9]

Compound 1 was prepared according to Example 4 (Steps 1 and 2 in FIG. 7).

### Step 3: Reduction with Sodium Borohydride

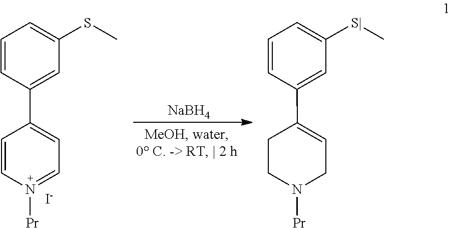

4-[3-(methylsulfanyl)phenyl]-1-propylpyridin-1-ium iodide (800 mg, 3.27 mmol, 1.0 eq) was dissolved in methanol (8.0 mL) and water (16.0 mL) and the solution was placed in an ice-cooled bath. NaBH₄ (743 mg, 19.64 mmol, 6 eq) was added and reaction mixture was left for 2 h at ambient temperature. After that, the mixture was quenched with 1N solution of HCl and extracted with DCM (×3). Organic phases were combined, dried over sodium sulphate, filtered and evaporated. Crude material was purified by flash column chromatography (DCM:MeOH, 9:1) to give 240 mg (Yield=28%) of 4-[3-(methylsulfanyl)phenyl]-1-propyl-1,2,3,6-tetrahydropyridine with 95% of LCMS purity. LCMS (ESI): exact mass for C15H21NS: 247.14; [M+H]⁺=248.05 found.

¹H NMR (300 MHz, DMSO-d₆) δ 7.39-7.18 (m, 4H), 6.23 (s, 1H), 3.98-3.75 (m, 2H), 3.17-3.06 (m, 2H), 2.83-2.70 (m, 2H), 2.51 (s, 3H), 1.79-1.61 (m, 2H), 0.94 (t, J=7.4 Hz, 3H).

*2H Overlapping with DMSO

### Step 4: Oxidation

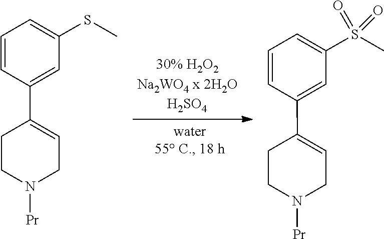

To a solution of 4-[3-(methylsulfanyl)phenyl]-1-propyl-1,2,3,6-tetrahydropyridine (240 mg, 0.97 mmol, 1.0 eq)) in water (12.0 mL), 96% H₂SO₄ (1.17 mL, 12.0 mmol, 12.37 eq) was added followed by sodium tungstate dihydrate (22 mg, 0.068 mmol, 0.07 eq) and 30% H₂O₂ (74 μL, 10.23 mmol, 2.42 eq). The reaction mixture was stirred at 55° C. for 2 h, after which time it was cooled to 10° C. and toluene (50 mL) was added followed by NaOH solution. The aqueous layer was extracted with toluene (3×). Organic layers were combined, dried over sodium sulphate and evaporated under reduced pressure to give 162 mg (Yield=58%) of 4-(3-methanesulfonylphenyl)-1-propyl-1,2,3,6-tetrahydropyridine as a yellow oil with 97% of LCMS purity. LCMS (ESI): exact mass for C15H21NO2S: 279.13; [M+H]⁺=280.05 found.

¹H NMR (300 MHz, DMSO-d₆) δ 7.95-7.87 (m, 1H), 7.83-7.73 (m, 2H), 7.66-7.55 (m, 1H), 6.36-6.28 (m, 1H), 3.23 (s, 3H), 3.14-3.06 (m, 2H), 2.68-2.59 (m, 2H), 2.41-2.30 (m, 2H), 1.58-1.43 (m, 2H), 0.88 (t, J=7.4 Hz, 3H). *2H overlapping with DMSO

### Step 5: Hydrogenation

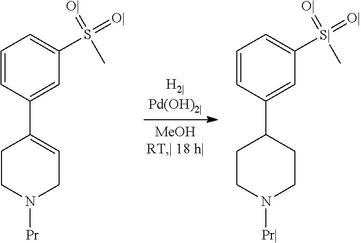

To a solution of 4-(3-methanesulfonylphenyl)-1-propyl-1,2,3,6-tetrahydropyridine (162 mg, 0.580 mmol, 1.0 eq) in methanol (8 mL) Pd(OH)₂/C (20% wt. loading, 50% wet, 16 mg, 0.012 mmol, 0.02 eq) was added under gentle flow of argon and the reaction mixture was evacuated and backfilled with hydrogen three times. Afterwards it was stirred under hydrogen atmosphere (balloon) overnight. Upon reaching completion, the mixture was filtered through the pad of Celite® and solvent was removed under reduced pressure. DCM (20 mL) and 2M NaOH solution (20 mL) were added and the mixture was stirred for 30 minutes at RT. Phases were separated and the water phase wase washed with DCM (2×). Organic phases were combined, dried over sodium sulphate, filtered and evaporated to give 88 mg (Yield=54%) of 4-(3-methanesulfonylphenyl)-1-propylpiperidine with 100% of LCMS purity which was purified in the next step during the HCl salt formation. LCMS (ESI): exact mass for C15H23NO2S: 281.14.; [M+H]⁺=282.10 found.

### Step 6: HCl Salt Formation

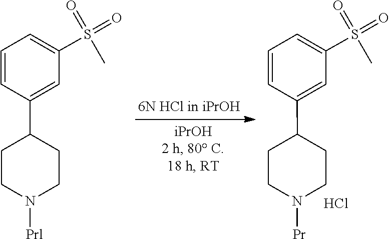

To a solution of 4-(3-(methylsulfonyl)phenyl)-1-propylpiperidine (88 mg, 0.313 mmol, 1.0 eq) was dissolved in iPrOH (4 mL), then 6N HCl in iPrOH was added dropwise (114 μl, 0.62 mmol, 2.0 eq). The reaction mixture was stirred 2 h at 80° C. and at room temperature overnight. The precipitate was filtered off, washed with iPrOH and dried under reduced pressure to give 43 mg (Yield=48%) of 4-(3-(methylsulfonyl)phenyl)-1-propylpiperidine hydrochloride as a pale yellow solid with 100% of LCMS purity. LCMS (ESI): exact mass for C15H23NO2S: 281.14; [M+H]⁺=281.65 found.

HPLC purity: 95.37% (@268 nm).

¹H NMR (300 MHz, Methanol-d₄) δ 7.92-7.83 (m, 2H), 7.70-7.58 (m, 2H), 3.78-3.66 (m, 2H), 3.22-3.02 (m, 8H), 2.25-1.94 (m, 4H), 1.91-1.74 (m, 2H), 1.06 (t, J=7.3 Hz, 3H).

### Example 7: Process for the Preparation of Pridopidine [FIG. 10]

### Step 1: Suzuki Coupling

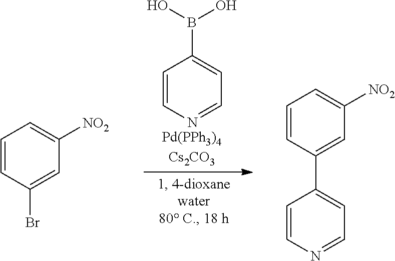 [SMILES: O=[N+]([O-])c1cccc(Br)c1 ; OB(O)c1ccncc1 ; O=[N+]([O-])c1cccc(-c2ccncc2)c1]

To a solution of 1-bromo-3-nitrobenzene (2.0 g, 9.90 mmol, 1.0 eq) in 1,4-dioxane (40 mL) and water (4 mL), pyridine-4-boronic acid (1.58 g, 12.87 mmol, 1.3 eq) and Cs₂CO₃ (9.68 g, 29.70 mmol, 3.0 eq) were added and reaction mixture was purged with argon for 15 minutes. Pd(PPh₃)₄ (1.14 g, 0.99 mmol, 0.1 eq) was added and the mixture was stirred at 80° C. overnight. Upon cooling to room temperature it was filtered through the pad of Celite®, washed with DCM and concentrated under reduced pressure. The residue was taken up in DCM, washed with water (3×), organic layer was dried over sodium sulphate, filtered and concentrated under reduced pressure. Crude material was purified by flash column chromatography (DCM:MeOH 100:0->90:10) to give 1.4 g (Yield=72%) of 4-(3-nitrophenyl)pyridine as a brown solid with 100% of LCMS purity. LCMS (ESI): exact mass for C11H8N2O2: 200.2; [M+H]⁺=201.0 found.

¹H NMR (300 MHz, Methanol-d₄) δ 8.68 (d, J=1.7 Hz, 1H), 8.67 (d, J=1.8 Hz, 1H), 8.61 (t, J=2.1 Hz, 1H), 8.39-8.31 (m, 1H), 8.23-8.14 (m, 1H), 7.85-7.74 (m, 3H).

### Step 2: N-propylation

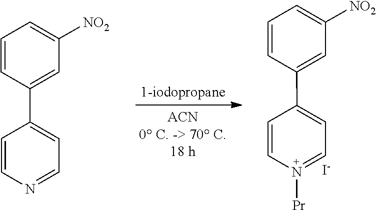

4-(3-Nitrophenyl)pyridine (1.3 g, 6.49 mmol, 1.0 eq) was dissolved in ACN (26 mL) and placed in an ice-cooled bath. 1-Iodopropane (1.27 mL, 12.99 mmol, 2.0 eq) was added dropwise and the reaction mixture was heated to 70° C. overnight. Upon cooling to room temperature, the reaction mixture was quenched with water, and extracted with EtOAc (×3). Combined organic layers were dried over anhydrous Na₂SO₄, filtered and concentrated under reduced pressure. Obtained solid was refluxed with EtOAc (30 mL) for 1h, cooled down to room temperature, washed with EtOAc and dried under reduced pressure to give 2.16 g (Yield=89%) of 4-(3-nitrophenyl)-1-propylpyridin-1-ium iodide as a yellow solid with 100% of LCMS purity. LCMS (ESI): exact mass for C14H15N2O2: 243.29; [M]+=242.85 found.

¹H NMR (300 MHz, Methanol-d₄) δ 9.15-9.08 (m, 2H), 8.90-8.84 (m, 1H), 8.58-8.50 (m, 3H), 8.46-8.38 (m, 1H), 7.93 (t, J=8.1 Hz, 1H), 4.67 (t, J=7.4 Hz, 2H), 2.13 (h, J=7.4 Hz, 2H), 1.08 (t, J=7.4 Hz, 3H).

### Step 3: Hydrogenation

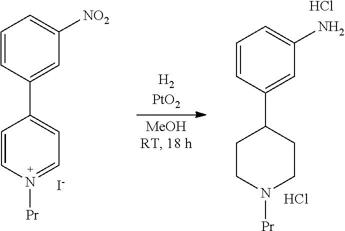

To a solution of 4-(3-nitrophenyl)-1-propylpyridin-1-ium iodide (1.0 g, 2.70 mmol, 1.0 eq) in methanol (40.0 mL) PtO₂ (307 mg, 1.35 mmol, 0.5 eq) was added under gentle flow of argon and the reaction mixture was evacuated and backfilled with hydrogen three times. Afterwards it was stirred under hydrogen atmosphere (balloon) overnight. Upon reaching completion, the mixture was filtered through the pad of Celite® and solvent was removed under reduced pressure. DCM (30 mL) and 2M NaOH solution (30 mL) were added and the mixture was stirred for 30 minutes at RT. Phases were separated and the water phase was washed with DCM (2×). Organic phases were combined, dried over sodium sulphate, filtered and concentrated under reduced pressure. The residue was taken up in methanol, 4N HCl in dioxane was added and the solution was evaporated under reduced pressure to give 1.05 g (Yield=quantitative) of 3-(1-propylpiperidin-4-yl)aniline dihydrochloride with 100% of LCMS purity. LCMS (ESI): exact mass for C14H22N2: 218.34; [M+H]⁺=219.10 found.

¹H NMR (300 MHz, Methanol-d₄) δ 7.04 (t, J=7.7 Hz, 1H), 6.66-6.55 (m, 3H), 3.44-3.34 (m, 2H), 2.84-2.75 (m, 2H), 2.76-2.54 (m, 3H), 2.01-1.84 (m, 4H), 1.78-1.65 (m, 2H), 1.00 (t, J=7.4 Hz, 3H).

### Step 4: Sandmeyer Reaction

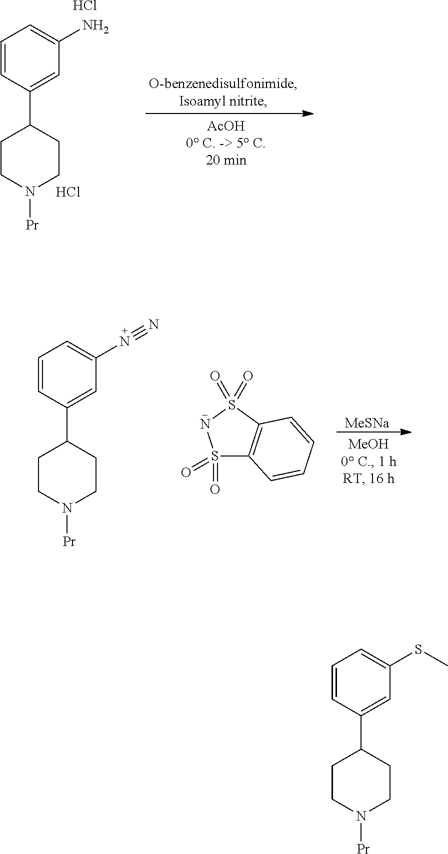

3-(1-Propylpiperidin-4-yl)aniline dihydrochloride (445 mg, 1.53 mmol, 1.0 eq) was dissolved in glacial AcOH (13 mL) and cooled to 0° C. in an ice bath. Solution of O-benzenedisulfonimide (536 mg, 2.45 mmol, 1.2 eq) in glacial AcOH (7 mL) was added over a period of 10 min, keeping the temperature below 5° C. Reaction mixture was stirred at 0° C. for 10 min, then isoamyl nitrite (302 μL, 2.24 mmol, 1.1 eq) was added dropwise over a period of 10 min. Solution was stirred for 20 min. at 0° C. and diethyl ether was added to precipitate the compound as an orange solid, which was filtered, washed with diethyl ether and dried under reduced pressure at RT. 3-(1-Propylpiperidin-4-yl)benzene-1-diazonium O-benzenedisulfonimide (914 mg, 2.04 mmol, 1.0 eq) was added in one portion to a solution of sodium methanethiolate (160 mg, 2.28 mmol, 1.12 eq) in MeOH (20 mL) at 0° C. Reaction mixture was stirred for 1h keeping the temperature below 5° C., then it was quenched with water (50 mL) and extracted with DCM (3×). Combined organic layers were dried over anhydrous Na₂SO₄, filtered and concentrated under reduced pressure. The crude material was purified by silica gel column chromatography (DCM:MeOH 100:0->85:15) followed by pTLC (CHCl₃:iPrOH, 9:1) to give 161 mg (Yield=30%) of 4-[3-(methylsulfanyl)phenyl]-1-propylpiperidine as yellow solid with 71% of LCMS purity. LCMS (ESI): exact mass for C15H23NS: 249.42; [M]+=250.10 found.

¹H NMR (300 MHz, DMSO-d₆) δ 7.32-7.15 (m, 2H), 7.13-6.95 (m, 2H), 2.99-2.89 (m, 2H), 2.46 (s, 3H), 2.30-2.16 (m, 2H), 2.01-1.82 (m, 2H), 1.81-1.53 (m, 5H), 1.52-1.34 (m, 2H), 0.86 (t, J=7.4 Hz, 3H).

### Step 5: Oxidation

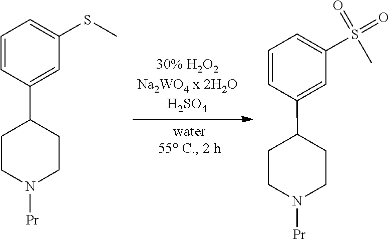

To a solution of 4-[3-(methylsulfanyl)phenyl]-1-propylpiperidine (140 mg, 0.56 mmol, 1.0 eq) in water (7 mL), 96% H₂SO₄ (370 μL, 6.94 mmol, 12.37 eq) was added followed by sodium tungstate dihydrate (13 mg, 0.04 mmol, 0.07 eq) and 30% H₂O₂ solution (144 μL, 1.40 mmol, 2.50 eq). The reaction mixture was stirred at 55° C. for 2 h, after which time it was cooled to 10° C. and toluene (50 mL) was added followed by NaOH solution. The aqueous layer was extracted with toluene (3×). Organic layers were combined, dried over sodium sulphate and evaporated under reduced pressure. The crude material was purified by pTLC (DCM:MeOH 9:1), to give 27 mg (Yield=17%) of 4-(3-methanesulfonylphenyl)-1-propylpiperidine as a yellow solid, with 56% of LCMS purity which was purified in the next step during the HCl salt formation. LCMS (ESI): exact mass for C15H23NO2S: 281.14; [M+H]⁺=282.00 found.

### Step 6: HCl Salt Formation

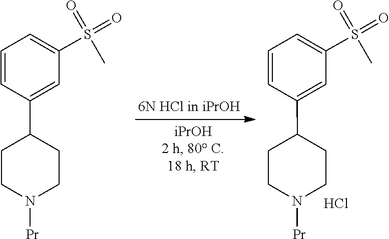

4-(3-methanesulfonylphenyl)-1-propylpiperidine (26 mg, 0.09 mmol, 1.0 eq) was dissolved in iPrOH (300 μL), heated up to 70° C. and 6N HCl in iPrOH (19 μL, 0.11 mmol, 1.2 eq) was added. The mixture was heated to 80° C., and stirred for 10 min. After this time it was cooled to 65° C., seeded with pure 4-(3-(methylsulfonyl)phenyl)-1-propylpiperidine hydrochloride salt and cooled to room temperature to precipitate the product. The white solid was filtered off, washed with iPrOH and dried under reduced pressure to give 11 mg (Yield=37%) of 4-(3-(methylsulfonyl)phenyl)-1-propylpiperidine hydrochloride as a white solid with 100% of LCMS purity. LCMS (ESI): exact mass for C15H23NO2S: 281.14; [M+H]⁺=282.05 found.

HPLC purity: 99.32% (@268 nm)

¹H NMR (300 MHz, Methanol-d₄) δ 7.92-7.84 (m, 2H), 7.74-7.59 (m, 2H), 3.76-3.61 (m, 2H), 3.23-2.99 (m, 8H), 2.28-1.94 (m, 4H), 1.89-1.74 (m, 2H), 1.06 (t, J=7.4 Hz, 3H).

### Example 8: Process for the Preparation of Pridopidine [FIG. 11]

### Step 1: Suzuki Coupling

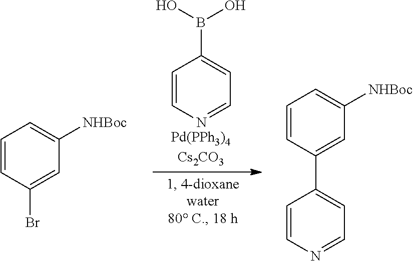 [SMILES: CC(C)(C)OC(=O)Nc1cccc(Br)c1 ; OB(O)c1ccncc1 ; CC(C)(C)OC(=O)Nc1cccc(-c2ccncc2)c1]

To a solution of tert-butyl N-(3-bromophenyl)carbamate (3.5 g, 12.86 mmol, 1.0 eq) in 1,4-dioxane (140 mL) and water (14 mL), pyridine-4-boronic acid (2.05 g, 16.71 mmol, 1.2 eq) and Cs₂CO₃ (12.57 g, 38.58 mmol, 3.0 eq) were added and reaction mixture was purged with argon for 15 minutes. Pd(PPh₃)₄ (1.48 g, 1.28 mmol, 0.1 eq) was added and the mixture was stirred at 80° C. overnight. After this time, the mixture was cooled to room temperature, filtered through the pad of Celite®, washed with DCM and concentrated under reduced pressure. The residue was taken up in DCM, washed with water (3×), organic layer was dried over sodium sulphate, filtered and concentrated under reduced pressure. Crude material was purified by silica column chromatography (DCM:MeOH 100:0->85:15) to give 3.1 g (Yield=quantitative) of tert-butyl N-[3-(pyridin-4-yl)phenyl]carbamate as a brown solid with 100% of LCMS purity. LCMS (ESI): exact mass for C16H18N2O2: 270.14; [M+H]⁺=270.60 found.

¹H NMR (300 MHz, DMSO-d₆) δ 9.51 (s, 1H), 8.68-8.59 (m, 2H), 7.94-7.87 (m, 1H), 7.67-7.58 (m, 2H), 7.55-7.48 (m, 1H), 7.47-7.36 (m, 2H), 1.49 (s, 9H).

### Step 2: N-propylation

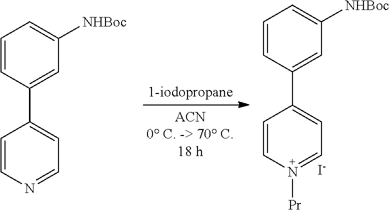

Tert-butyl N-[3-(pyridin-4-yl)phenyl]carbamate (3.0 g, 11.08 mmol, 1.0 eq) was dissolved in ACN (60 mL) and placed in an ice-cooled bath. 1-Iodopropane (4.32 mL, 44.39 mmol, 4.0 eq) was added dropwise and the reaction mixture was heated to 70° C. overnight. Upon cooling to room temperature, the reaction mixture was quenched with water, and extracted with EtOAc (×3). Combined organic layers were dried over anhydrous Na₂SO₄, filtered and concentrated under reduced pressure. Obtained solid was refluxed with EtOAc (10 mL) for 1h, cooled down to room temperature, washed with EtOAc and dried under reduced pressure to give 3.5 g (Yield=72%) of 4-(3-{[(tert-butoxy)carbonyl]amino}phenyl)-1-propylpyridin-1-ium iodide as a yellow solid with 100% of LCMS purity. LCMS (ESI): exact mass for C19H25N2O2: 313.19; [M+H]⁺=313.9 found.

¹H NMR (300 MHz, DMSO-d₆) δ 9.66 (s, 1H), 9.15-9.05 (m, 2H), 8.41-8.34 (m, 2H), 8.17-8.12 (m, 1H), 7.72-7.47 (m, 3H), 4.57 (t, J=7.3 Hz, 2H), 1.96 (h, J=7.3 Hz, 2H), 1.50 (s, 9H), 0.92 (t, J=7.4 Hz, 3H).

### Step 3: Hydrogenation

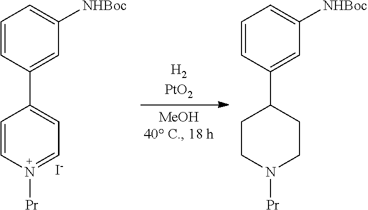

To a solution of 4-(3-{[(tert-butoxy)carbonyl]amino}phenyl)-1-propylpyridin-1-ium iodide (1.0 g, 2.27 mmol, 1.0 eq) in methanol (50 mL) PtO₂ (362 mg, 1.59 mmol, 0.7 eq) was added under gentle flow of argon and the reaction mixture was evacuated and backfilled with hydrogen three times. Afterwards it was stirred under hydrogen atmosphere (balloon) overnight at 40° C. Upon reaching completion, the mixture was filtered through the pad of Celite® and solvent was removed under reduced pressure to give 1.1 g (Yield=quantitative) of tert-butyl N-[3-(1-propylpiperidin-4-yl)phenyl]carbamate with 100% of LCMS purity. LCMS (ESI): exact mass for C19H30N2O2: 318.23; [M+H]⁺=319.15 found.

¹H NMR (300 MHz, Methanol-d₄) δ 7.43 (s, 1H), 7.24-7.12 (m, 2H), 6.95-6.82 (m, 1H), 3.63-3.50 (m, 2H), 3.07-2.92 (m, 4H), 2.90-2.75 (m, 1H), 2.16-1.87 (m, 4H), 1.85-1.67 (m, 2H), 1.51 (s, 9H), 1.02 (t, J=7.3 Hz, 3H).

### Step 3.1: Reduction with NaBH₄

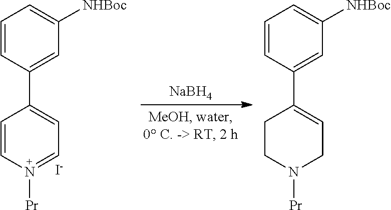

A solution of 4-(3-{[(tert-butoxy)carbonyl]amino}phenyl)-1-propylpyridin-1-iumiodide (1.0 g, 2.27 mmol, 1.0 eq) was dissolved in methanol (10 mL) and water (20 mL) and placed in an ice-cooled bath. NaBH₄ (724 mg, 19.14 mmol, 8.4 eq) was added and reaction mixture was left for 2 h. After that, the mixture was quenched with 1N solution of HCl and extracted with DCM (×3). Organic phases were combined, dried over sodium sulphate, filtered and evaporated to give 815 mg (Yield=quantitative) of tert-butyl N-[3-(1-propyl-1,2,3,6-tetrahydropyridin-4-yl)phenyl]carbamate with 91% of LCMS purity which was used as is in the next step. LCMS (ESI): exact mass for C19H28N2O2: 316.22; [M+H]⁺=316.65 found.

¹H NMR (300 MHz, Methanol-d₄) δ 7.60 (s, 1H), 7.31-7.19 (m, 2H), 7.14-7.04 (m, 1H), 6.17-6.11 (m, 1H), 3.72-3.65 (m, 2H), 3.30-3.26 (m, 2H), 3.01-2.90 (m, 2H), 2.85-2.69 (m, 2H), 1.87-1.68 (m, 2H), 1.52 (s, 9H), 1.03 (t, J=7.4 Hz, 3H).

### Step 3.2: Hydrogenation

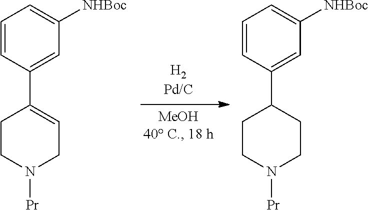

To a solution of tert-butyl N-[3-(1-propyl-1,2,3,6-tetrahydropyridin-4-yl)phenyl]carbamate (800 mg, 3.23 mmol, 1.0 eq) in methanol (80 mL) 10% Pd/C (60-65% wet, 229 mg, 1.13 mmol, 0.35 eq) was added under gentle flow of argon and the reaction mixture was evacuated and backfilled with hydrogen three times. Afterwards it was stirred under hydrogen atmosphere (balloon) overnight at 40° C. Upon reaching completion, the mixture was filtered through the pad of Celite® and solvent was removed under reduced pressure to give 470 mg (Yield=45%) of tert-butyl N-[3-(1-propylpiperidin-4-yl)phenyl]carbamate with 100% of LCMS purity. LCMS (ESI): exact mass for C19H30N2O2: 318.23; [M+H]⁺=319.15 found.

¹H NMR (300 MHz, Methanol-d₄) δ 7.44 (s, 1H), 7.27-7.12 (m, 2H), 7.00-6.82 (m, 1H), 3.66-3.48 (m, 2H), 3.12-2.93 (m, 4H), 2.93-2.72 (m, 1H), 2.18-1.88 (m, 4H), 1.87-1.67 (m, 2H), 1.52 (s, 9H), 1.04 (t, J=7.4 Hz, 3H).

### Step 4: BOC-deprotection

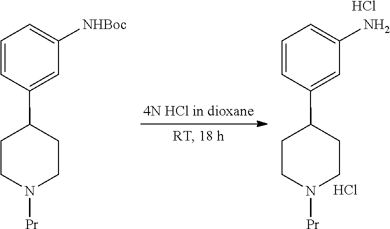

(470 mg, 1.47 mmol, 1.0 eq) was dissolved in dioxane (36 mL). 4N HCl in dioxane (10.4 mL, 43.71 mmol, 29.7 eq) was added dropwise and the reaction mixture was stirred at RT for 18 h. The solution was concentrated under reduced pressure to give 416 mg (Yield=97%) of 3-(1-propylpiperidin-4-yl)aniline dihydrochloride as a brown solid with 100% of LCMS purity. LCMS (ESI): exact mass for C14H22N2: 218.18; [M+H]⁺=218.75 found.

¹H NMR (300 MHz, Methanol-d₄) δ 7.53 (t, J=7.8 Hz, 1H), 7.49-7.41 (m, 1H), 7.39-7.34 (m, 1H), 7.33-7.27 (m, 1H), 3.77-3.65 (m, 2H), 3.23-2.96 (m, 5H), 2.25-1.98 (m, 4H), 1.94-1.69 (m, 2H), 1.05 (t, J=7.4 Hz, 3H).

### Step 5: Sandmeyer Reaction

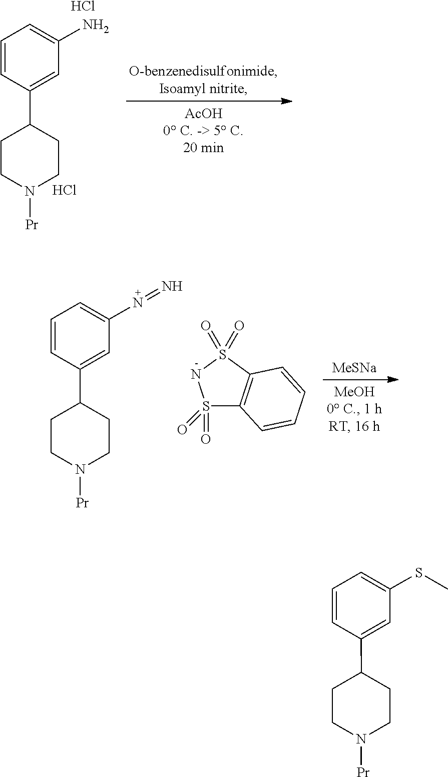

3-(1-Propylpiperidin-4-yl)aniline dihydrochloride (445 mg, 1.53 mmol, 1.0 eq) was dissolved in glacial AcOH (13 mL) and cooled to 0° C. in an ice bath. Solution of 0-benzenedisulfonimide (536 mg, 2.45 mmol, 1.2 eq) in glacial AcOH (7 mL) was added over a period of 10 min, keeping the temperature below 5° C. Reaction mixture was stirred at 0° C. for 10 min, then isoamyl nitrite (302 μL, 2.24 mmol, 1.1 eq) was added dropwise over a period of 10 min. Solution was stirred for 20 min. at 0° C. and diethyl ether was added to precipitate the compound as an orange solid, which was filtered, washed with diethyl ether and dried under reduced pressure at RT. 3-(1-Propylpiperidin-4-yl)benzene-1-diazonium O-benzenedisulfonimide (914 mg, 2.04 mmol, 1.0 eq) was added in one portion to a solution of sodium methanethiolate (160 mg, 2.28 mmol, 1.12 eq) in MeOH (20 mL) at 0° C. Reaction mixture was stirred for 1h keeping the temperature below 5° C., then it was quenched with water (50 mL) and extracted with DCM (3×). Combined organic layers were dried over anhydrous Na2SO4, filtered and concentrated under reduced pressure. The crude material was purified by silica gel column chromatography (DCM:MeOH 100:0->85:15) followed by pTLC (CHCl₃:iPrOH, 9:1) to give 161 mg (Y=30%) of 4-[3-(methylsulfanyl)phenyl]-1-propylpiperidine as yellow solid with 71% of LCMS purity. LCMS (ESI): exact mass for C15H23NS: 249.42; [M]+=250.10 found.

¹H NMR (300 MHz, DMSO-d₆) δ 7.32-7.15 (m, 2H), 7.13-6.95 (m, 2H), 2.99-2.89 (m, 2H), 2.46 (s, 3H), 2.30-2.16 (m, 2H), 2.01-1.82 (m, 2H), 1.81-1.53 (m, 5H), 1.52-1.34 (m, 2H), 0.86 (t, J=7.4 Hz, 3H).

### Step 6: Oxidation

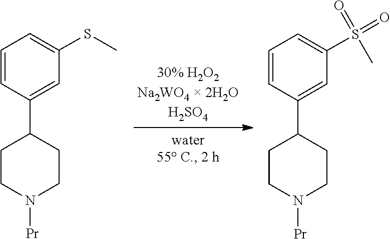

To a solution of 4-[3-(methylsulfanyl)phenyl]-1-propylpiperidine (140 mg, 0.56 mmol, 1.0 eq) in water (7 mL), 96% H₂SO₄ (370 μL, 6.94 mmol, 12.37 eq) was added followed by sodium tungstate dihydrate (13 mg, 0.04 mmol, 0.07 eq) and 30% H₂O₂ solution (144 μL, 1.40 mmol, 2.50 eq). The reaction mixture was stirred at 55° C. for 2 h, after which time it was cooled to 10° C. and toluene (50 mL) was added followed by NaOH solution. The aqueous layer was extracted with toluene (3×). Organic layers were combined, dried over sodium sulphate and evaporated under reduced pressure. The crude material was purified by pTLC (DCM:MeOH 9:1), to give 27 mg (Y=17%) of 4-(3-methanesulfonylphenyl)-1-propylpiperidine as a yellow solid, with 56% of LCMS purity which was purified in the next step during the HCl salt formation. LCMS (ESI): exact mass for C15H23NO2S: 281.14; [M+H]⁺=282.00 found.

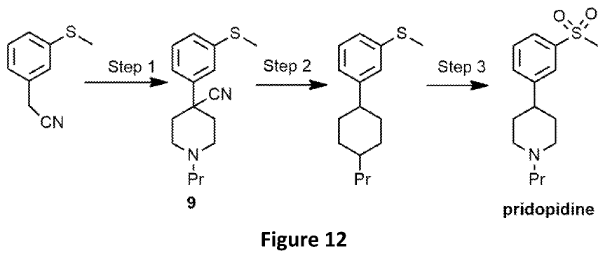

### Example 9: Process for the Preparation of Pridopidine [FIG. 12]

### Step 1: Cyclization Reaction

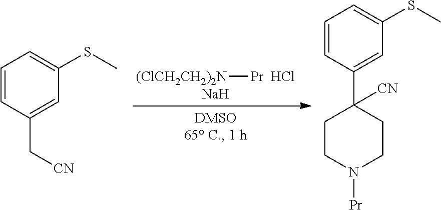

A solution of (3-methylsulfanyl-phenyl)-acetonitrile (2.0 g, 12.3 mmol, 1.0 eq) in DMSO (16 mL) was treated with sodium hydride (60% dispersion, mineral oil, 1.47 g, 36.8 mmol, 3.0 eq) portion-wise. The resulting suspension was stirred at RT for 30 minutes. After this time N,N-bis(2-chloroethyl)propan-1-amine hydrochloride (2.97 g, 13.5 mmol, 1.1 eq) was slowly added over 5 minutes and the suspension was heated at 65° C. for 1h. Upon completion, the reaction was diluted with water and extracted with ethyl acetate (3×). The combined extracts were washed with water (5×), dried over anhydrous sodium sulphate, filtered and concentrated under reduced pressure to give 3.44 g (Yield=94%) of 4-[3-(methylsulfanyl)phenyl]-1-propylpiperidine-4-carbonitrile as a brown oil with 92% purity. LCMS (ESI): exact mass for C16H22N2S: 274.15; [M+H]⁺=275.05 found.

¹H NMR (300 MHz, Methanol-d₄) δ 7.47-7.41 (m, 2H), 7.32-7.27 (m, 2H), 3.12-3.04 (m, 2H), 2.49-2.40 (m, 7H), 2.15-2.07 (m, 4H), 1.57 (m, 2H), 0.95 (t, 3H).

### Step 2: Hydrolysis-decarboxylation

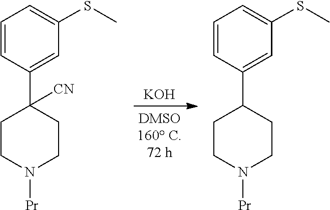

4-[3-(methylsulfanyl)phenyl]-1-propylpiperidine-4-carbonitrile (2.9 g, 10.6 mmol, 1.0 eq) was dissolved in DMSO (58 mL) and potassium hydroxide (5.93 g, 105.7 mmol, 10.0 eq) was added portion-wise. The reaction mixture was heated to 160° C. and stirred for 72h. Upon cooling to room temperature, the reaction mixture was quenched with water, and extracted with DCM (3×). The combined extracts were washed with water (5×), dried over anhydrous sodium sulphate, filtered and concentrated under reduced pressure. The crude material was purified using flash column chromatography (DCM:DCM/MeOH 9:1+1% NH₃) to give 223 mg (Yield=8%) of 4-[3-(methylsulfanyl)phenyl]-1-propylpiperidine as a brown solid with 98% of LCMS purity. LCMS (ESI): exact mass for C15H23NS: 249.16; [M+H]⁺=250.10 found.

¹H NMR (300 MHz, Methanol-d₄) δ 7.23-7.15 (m, 4H), 3.13 (d, 2H), 2.47-2.40 (m, 5H), 2.26-2.14 (m, 2H), 1.90-1.53 (m, 7H), 0.95 (t, J=7.4 Hz, 3H).

Note: During the process optimization the reaction was successfully run on a 50 mg scale resulting in 41 mg (Yield=91%) of the product with 80% by LCMS purity.

### Step 3: Oxidation

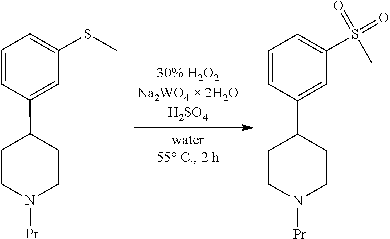

To a solution of 4-[3-(methylsulfanyl)phenyl]-1-propylpiperidine (200 mg, 0.80 mmol, 1.0 eq) in water (10 mL), 96% H₂SO₄ (528 μL, 9.9 mmol, 12.37 eq) was added followed by sodium tungstate dihydrate (20 mg, 0.10 mmol, 0.07 eq) and 30% H₂O₂ (200 μL, 2.0 mmol, 2.50 eq). The reaction mixture was stirred at 55° C. for 2 h. After this time, the mixture was cooled to 10° C. and toluene followed by NaOH solution was added until pH was ˜12. The aqueous layer was extracted with toluene (3×). Organic layers were combined, dried over sodium sulphate and evaporated under reduced pressure to give 192 mg (Yield=85%) of 4-(3-methanesulfonylphenyl)-1-propylpiperidine as a brown oil, with 92% by LCMS purity which was purified in the next step during the HCl salt formation. LCMS (ESI): exact mass for C15H23NO2S: 281.14; [M+H]⁺=282.05 found.

### Step 4: Salt formation

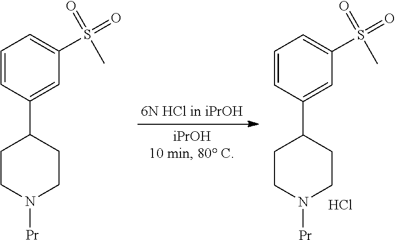

4-(3-methanesulfonylphenyl)-1-propylpiperidine (192 mg, 0.68 mmol, 1.0 eq) was taken up in iPrOH (2.1 mL), heated up to 70° C. and 6N HCl in iPrOH (136 μL, 0.82 mmol, 1.2 eq) was added. The mixture was heated to 80° C., and stirred for 10 min. After this time it was cooled to 65° C. and seeded with pure 4-(3-(methylsulfonyl)phenyl)-1-propylpiperidine hydrochloride salt and cooled to room temperature. Upon cooling the solution, white precipitate was formed which was washed with iPrOH and dried to give 73 mg (Yield=34%) of 4-(3-(methylsulfonyl)phenyl)-1-propylpiperidine hydrochloride as a white solid with 100% of LCMS purity. LCMS (ESI): exact mass for C15H23NO2S: 281.14; [M+H]⁺=282.10 found.

HPLC purity: 94.40% (@268 nm)

¹H NMR (300 MHz, Methanol-d₄) δ 8.00-7.90 (m, 2H), 7.62-7.53 (m, 2H), 3.78-3.66 (m, 2H), 3.23-3.00 (m, 8H), 2.23-1.95 (m, 4H), 1.94-1.75 (m, 2H), 1.06 (t, J=7.4 Hz, 3H).

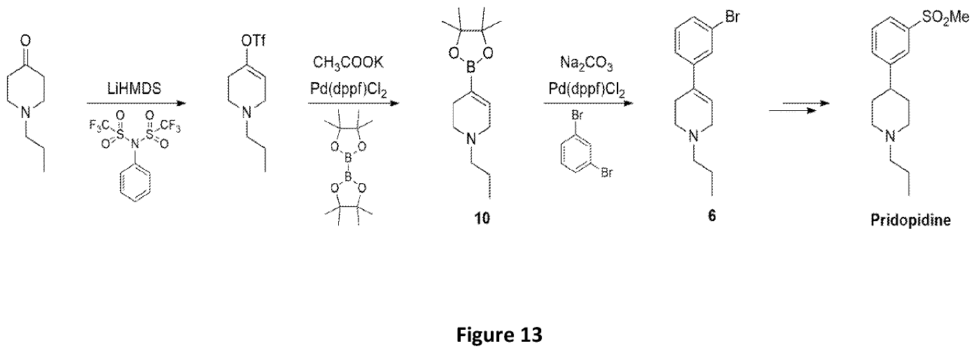

### Example 10: Process for the Preparation of Pridopidine [FIG. 13]

### Step 1: Triflate formation

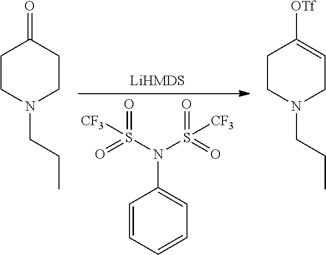

### 1-Propyl-1,2,3,6-tetrahydropyridin-4-yl trifluoromethanesulfonate

A clear brown solution of 1-propylpiperidin-4-one (506 mg, 3.58 mmol) in THE (2.5 mL) was cooled to −75° C. LiHMDS (779 mg, 4.66 mL, 1M, 1.3 Eq, 4.66 mmol) was added dropwise keeping the temperature below −70° C. The reaction mixture was stirred for 30 min at −75° C. NFSI (1.41 g, 1.1 Eq, 3.94 mmol) was added portion wise. After addition the reaction mixture was allowed to warm to room temperature and stirrer overnight. The reaction mixture was diluted with EtOAc (5 mL). The reaction mixture was cooled using an ice bath. Sat. aq. NH₄Cl (5 mL) was added dropwise. The phases were separated and the organic phase was washed with aq. NH₄Cl (5 mL) and water (5 mL). The organic phase was dried over Na₂SO₄ and concentrated in vacuo to afford 1.84 g. The material was purified by the means of column chromatography (silica; 0-3% 7N NH₃ in MeOH in DCM): Fractions 3 (tubes 37-43) was concentrated in vacuo to afford 1-propyl-1,2,3,6-tetrahydropyridin-4-yl trifluoromethanesulfonate as a light-yellow oil (626 mg, 2.29 mmol, Yield=63.9%).

### Step 2: Boronic Ester Formation

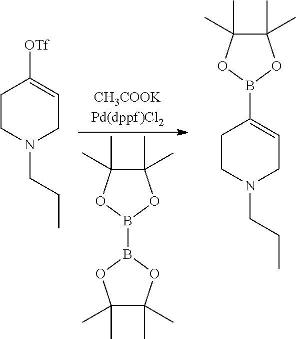

### 1-Propyl-4-(4,4,5,5-tetramethyl-1,3,2-dioxaborolan-2-yl)-1,2,3,6-tetrahydropyridine (Compound 10)

A suspension of 1-propyl-1,2,3,6-tetrahydropyridin-4-yl trifluoromethanesulfonate (1.95 g, 7.14 mmol), KOAc (2.10 g, 3 Eq, 21.4 mmol) and B₂Pin₂ (2.17 g, 1.2 Eq, 8.56 mmol) in 1,4-dioxane (22 mL) was stirred at room temperature. PdCl₂(dppf) (261 mg, 0.05 Eq, 357 μmol) was added and the reaction mixture was degassed with N₂ for 15 min. Afterwards the reaction mixture was heated to 80° C. for 2.5 hours. The reaction mixture was filtered over a layer of Celite. The filter cake was washed with 1,4-dioxane. The combined filtrates were concentrated in vacuo to afford crude 1-propyl-4-(4,4,5,5-tetramethyl-1,3,2-dioxaborolan-2-yl)-1,2,3,6-tetrahydropyridine (4.25 g, 6.1 mmol, Yield=85%, 36% Purity based on H-QNMR). The material was used as such without further purification.

### Step 3: Suzuki Miyaura Reaction

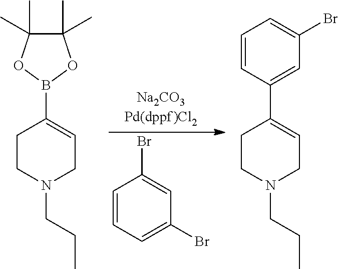

### 4-(3-Bromophenyl)-1-propyl-1,2,3,6-tetrahydropyridine (Compound 6)

1-Propyl-4-(4,4,5,5-tetramethyl-1,3,2-dioxaborolan-2-yl)-1,2,3,6-tetrahydropyridine (615 mg, 36% Wt, 881 μmol) was dissolved in 1,2-dimethoxyethane (10 mL). 1,3-Dibromobenzene (1.04 g, 533 μL, 5 Eq, 4.41 mmol), followed by Na₂CO₃ (280 mg, 1.32 mL, 2 molar, 3 Eq, 2.64 mmol) and PdCl₂(dppf) (32.2 mg, 0.05 Eq, 44.1 μmol) were added. The reaction mixture was degassed for 15 minutes with N₂ and stirred at 80° C. for 3 hours. EtOAc (100 mL) and water (50 mL) were added. The mixture was filtered over a layer of Celite. The phases were separated, and the organic phase was washed with water (2×50 mL). The organic phase was dried over Na₂SO₄ and concentrated in vacuo to afford 1 g crude. The crude product was purified by the means of column chromatography (silica; 0-3% 7N NH₃ in MeOH in DCM): Fraction 3 (tubes 21-34) was concentrated in vacuo to afford 4-(3-bromophenyl)-1-propyl-1,2,3,6-tetrahydropyridine (95 mg, 0.34 mmol, Yield=38%).

Chemistry to convert the alkene bromo intermediate towards Pridopidine in analogy to the described above in Example 1. First reduction of the double bound of Compound 6 to obtain compound 8 which is further converted to Pridopidine.

### Example 11: Process for the Preparation of Pridopidine

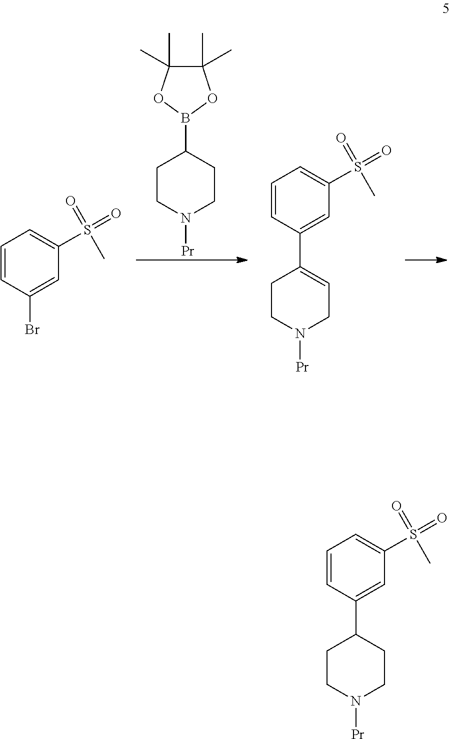

Boronic ester (Compound 10) was synthesized as described in Example 10 and further reacted with 1-bromo-3-(methylsulfonyl)benzene to give Compound 5. The double bond of Compound 5 was reduced as described in Example 6 step 5 towards pridopidine.

### Step 1: Suzuki Coupling

To a solution of 1-bromo-3-(methylsulfonyl)benzene (0.415 g, 1.76 mmol, 1.0 eq) in 1,4-dioxane (5 mL) and water (1 mL), N-propyl-2H-pyridine-4-boronic acid pinacol ester (0.53 g, 2.11 mmol, 1.2 eq) and potassium acetate (0.52 g, 5.28 mmol, 3.0 eq) were added and reaction mixture was purged with argon for 15 minutes. Pd(dppf)Cl₂ (66 mg, 0.09 mmol, 0.05 eq) was added and the mixture was stirred at 80° C. overnight. After this time, the mixture was cooled to room temperature, filtered through the pad of Celite®, washed with DCM and concentrated under reduced pressure. The residue was taken up in DCM, washed with water (3×), organic layer was dried over sodium sulphate, filtered and concentrated under reduced pressure to give 0.18 g (Yield=37%) of 4-(3-methanesulfonylphenyl)-1-propyl-1,2,3,6-tetrahydropyridine as an oil after chromatographic purification LCMS (ESI): exact mass for C15H21NO2S: 279.13; [M+H]⁺=280.05 found.

### Step 2: Reduction with Formic Acid

To a cold solution of 4-(3-methanesulfonylphenyl)-1-propyl-1,2,3,6-tetrahydropyridine (1 g, 3.58 mmol, 1 eq) in 2.5 volumes of water formic acid (0.264 g, 5.73 mmol 1.6 eq and 0.1% (w/w) 100% Pd/C are added. The reaction is heated to 30° C. for about 4-5 hours. The mixture is filtered to remove the catalyst, the filtrate is added to toluene and the mixture is basified with dilute NaOH solution. The lower aqueous phase is separated, and the toluene phase is washed a few times with 5 volumes of water remove residual NaOH. The toluene is distilled off under vacuum, to the residue 4 volumes of n-heptane are added to form a slurry which is cooled down to 0° C. The solid material formed is collected by filtration and dried under vacuum at 40° C. The 4-(3-(methylsulfonyl)phenyl)-1-propylpiperidine obtained, Yield=85%.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1-23. (canceled)

24. A compound represented by the structure of Compound 1: 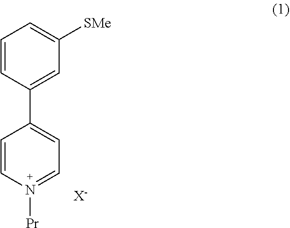
wherein X⁻ is an anion.

25. A process for the preparation of pridopidine wherein the process is via Compound 1 of claim 24.

26. The process of claim 25, wherein the process comprises oxidizing the —SMe to —SO₂Me of Compound 1 followed by reducing the pyridinium ring to obtain pridopidine.

27. The process of claim 26, wherein the reduction of the pyridinium ring comprises a reaction with PtO₂ and H₂ (gas).

28. The process of claim 25, wherein the process (Process 1) comprises:
reducing a pyridinium group of Compound 1 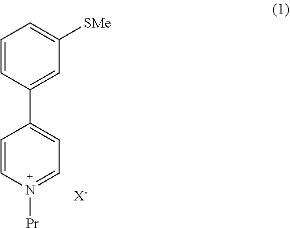
wherein X⁻ is an anion;
to obtain Compound 7 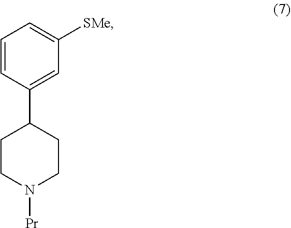
wherein, Compound 7 is further oxidized (—SMe to —SO₂Me) to obtain pridopidine.

29. The process of claim 28, wherein the reduction of the pyridinium ring comprises a reaction with PtO₂ and H₂ (gas).

30. The process of claim 26, wherein the oxidation of —SMe to —SO₂Me comprises a reaction with tungsten catalytic oxidizing agent, peroxide or combination thereof.

31. The process of claim 30, wherein the tungsten catalytic oxidizing agent is sodium tungstate.

32. The process of claim 30, wherein the peroxide is sodium peroxide.

33. The process of claim 25, wherein the process (Process 2) comprises:
reducing a pyridinium group of Compound 1: 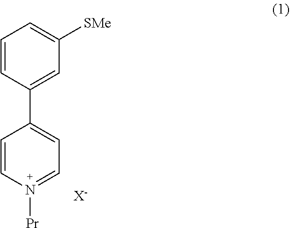
wherein X⁻ is an anion;
to obtain Compound 4, 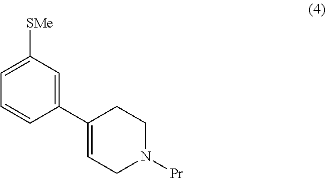
wherein,
Compound 4 is further oxidized (—SMe to —SO₂Me) and then reduced (the double bond) to obtain pridopidine; or alternatively, Compound 4 is further reduced (the double bond) and then oxidized (—SMe to —SO₂Me) to obtain pridopidine.

34. The process of claim 33, wherein the reducing agent for the reduction of the pyridinium ring of Compound 1, to obtain Compound 4 comprises sodium borohydride, sodium cyanoborohydride, sodium triacetoxyborohydride, or H₂ (gas) in the presence of Pd/C catalyst.

35. The process of claim 33, wherein the reduction of the double bond to obtain a piperidine ring comprises a reaction with a hydrogen source and a catalyst, wherein the catalyst comprises a second palladium catalyst, a platinum catalyst or a ruthenium catalyst.

36. The process of claim 35, wherein the hydrogen source comprises hydrogen gas, formic acid or a salt of formic acid.

37. The process of claim 36, wherein the salt of formic acid is ammonium format.

38. The process of claim 33, wherein the oxidation of —SMe to —SO₂Me comprises a reaction with tungsten catalytic oxidizing agent, peroxide or combination thereof.

39. The process of claim 38, wherein the tungsten catalytic oxidizing agent is sodium tungstate.

40. The process of claim 38, wherein the peroxide is sodium peroxide.

41. A process for the preparation of Compound 1: 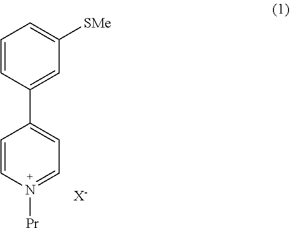
wherein X is an anion;
wherein the process (Process 3) comprises:
a) reacting a compound of Formula II: 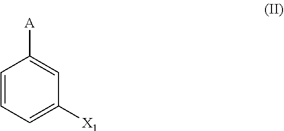
wherein A is SMe; and X₁ is halide;
with 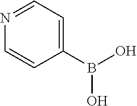
(pyridin-4-ylboronic acid) in the presence of a palladium catalyst and a weak base, to obtain a compound of Formula III 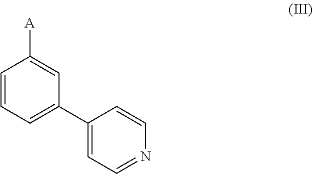
wherein A is SMe; and
b) reacting the compound of Formula III with a propyl moiety to obtain Compound 1.

42. The process of claim 41, wherein the weak base comprises potassium carbonate, potassium phosphate, sodium bicarbonate, sodium ethoxide and cesium carbonate or any combination thereof.

43. The process of claim 41, wherein the palladium catalyst comprises Tetrakis(triphenylphosphine)palladium(0) (Pd(Ph₃P)₄), Palladium(II) acetate (Pd(OAc)₂), Bis(triphenylphosphine)palladium chloride (Pd(Ph₃P)₂Cl₂), or [1,1′-Bis(diphenylphosphino)ferrocene]palladium(II) dichloride (Pd(Cl₂)dppf).

44. The process of claim 41, wherein the propyl moiety is 1-iodopropane.

45. A process for the preparation of pridopidine using Compound 1, 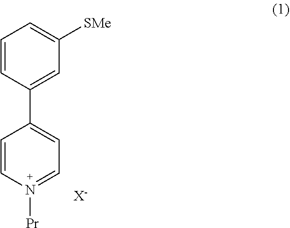
wherein X⁻ is an anion;
wherein Compound 1 is prepared according to claim 41.

46. The process for the preparation of pridopidine according to claim 28, wherein the process for the preparation of Compound 1 comprises:
a) reacting a compound of Formula II: 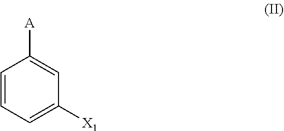
wherein A is SMe; and X₁ is halide;
with 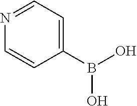
(pyridin-4-ylboronic acid) in the presence of a palladium catalyst and a weak base, to obtain a compound of Formula III 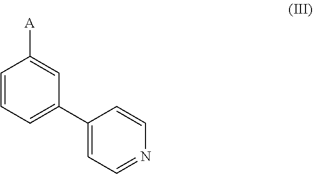
wherein A is SMe; and
b) reacting the compound of Formula III with a propyl moiety to obtain Compound 1.

47. The process for the preparation of pridopidine according to claim 33, wherein the process for the preparation of Compound 1 comprises:
a) reacting a compound of Formula II: 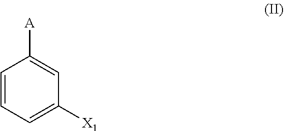
wherein A is SMe; and X₁ is halide;
with 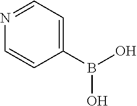
(pyridin-4-ylboronic acid) in the presence of a palladium catalyst and a weak base, to obtain a compound of Formula III 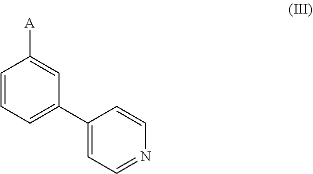
wherein A is SMe; and
b) reacting the compound of Formula III with a propyl moiety to obtain Compound 1.
